(19) United States
(12) Patent Application Publication
(10) Pub. No.: US 2012/0288568 A1
(43) Pub. Date: Nov. 15, 2012

(54) METHODS AND COMPOSITIONS FOR TREATING OPHTHALMIC CONDITIONS VIA SERUM RETINOL, SERUM RETINOL BINDING PROTEIN (RBP), AND/OR SERUM RETINOL-RBP MODULATION

(75) Inventors: Kenneth Widder, San Diego, CA (US); Jay Lichter, San Diego, CA (US); Nathan L. Mata, San Diego, CA (US)

(73) Assignee: Revision Therapeutics, Inc., La Jolla, CA (US)

(21) Appl. No.: 13/474,582

(22) Filed: May 17, 2012

Related U.S. Application Data
(63) Continuation of application No. 11/484,228, filed on Jul. 10, 2006, now abandoned.
(60) Provisional application No. 60/698,512, filed on Jul. 11, 2005.

(51) Int. Cl. A61K 31/167; A61K 31/661; A61P 9/00; A61P 27/02; A61P 29/00; A61K 33/00; A61K 31/203

(52) U.S. Cl. 424/600; 514/613; 514/121; 514/559

(57) ABSTRACT

Compounds that reduce serum retinol, serum RBP, and/or serum retinol-RBP levels may be used to treat ophthalmic conditions associated with the overproduction of waste products that accumulate during the course of the visual cycle. We describe methods and compositions using such compounds and their derivatives to treat, for example, the macular degenerations and dystrophies or to alleviate symptoms associated with such ophthalmic conditions. Such compounds and their derivatives may be used as single agent therapy or in combination with other agents or therapies.

### RELATED APPLICATIONS

This patent application claims the benefit of U.S. Provisional Patent Application No. 60/698,512 filed Jul. 11, 2005. This patent application is related to U.S. patent application Ser. Nos. 11/150,641, filed Jun. 10, 2005; 11/296,909, filed Dec. 7, 2005; and 11/267,395 filed Nov. 4, 2005, all of which are herein incorporated by reference in their entirety.

### FIELD OF THE INVENTION

The methods and compositions described herein are directed to the treatment of ophthalmic conditions.

### BACKGROUND OF THE INVENTION

The visual cycle or retinoid cycle is a series of light-driven and enzyme catalyzed reactions in which the active visual chromophore rhodopsin is converted to an all-trans-isomer that is then subsequently regenerated. Part of the cycle occurs within the outer segment of the rods and part of the cycle occurs in the retinal pigment epithelium (RPE). Components of this cycle include various dehydrogenases and isomerases, as well as proteins for transporting intermediates between the photoreceptors and the RPE.

Other proteins associated with the visual cycle are responsible for transporting, removing and/or disposing of compounds and toxic products that accumulate from excess production of visual cycle retinoids, such as all-trans-retinal (atRAL). For example, N-retinylidene-N-retinylethanolamine (A2E) arises from the condensation of all-trans-retinal with phosphatidylethanolamine. Although certain levels of this orange-emitting fluorophore are tolerated by the photoreceptors and the RPE, excessive quantities can lead to adverse effects, including the production of lipofuscin, and potentially drusen under the macula. See, e.g., Finnemann, S. C., Proc. Natl. Acad. Sci., 99:3842-47 (2002). In addition, A2E can be cytotoxic to the RPE, which can lead to retinal damage and destruction. Drusen are extracellular deposits that accumulate below the RPE and are risk factors for developing age-related macular degeneration. See, e.g., Crabb, J. W., et al., Proc. Natl. Acad. Sci., 99:14682-87 (2002). Thus, removal and disposal of toxic products that arise from side reactions in the visual cycle are important because several lines of evidence indicate that the over-accumulation of toxic products is partially responsible for the symptoms associated with the macular degenerations and retinal dystrophies.

There are two general categories of age-related macular degeneration: the wet and dry forms. Dry macular degeneration, which accounts for about 90 percent of all cases, is also known as atrophic, nonexudative, or drusenoid macular degeneration. With dry macular degeneration, drusen typically accumulate beneath the RPE tissue in the retina. Vision loss can then occur when drusen interfere with the function of photoreceptors in the macula. This form of macular degeneration results in the gradual loss of vision over many years.

Wet macular degeneration, which accounts for about 10 percent of cases, is also known as choroidal neovascularization, subretinal neovascularization, exudative, or disciform degeneration. In wet macular degeneration, abnormal blood vessel growth can form beneath the macula; these vessels can leak blood and fluid into the macula and damage photoreceptor cells. Studies have shown that the dry form of macular degeneration can lead to the wet form of macular degeneration. The wet form of macular degeneration can progress rapidly and cause severe damage to central vision.

Stargardt Disease, also known as Stargardt Macular Dystrophy or Fundus Flavimaculatus, is the most frequently encountered juvenile onset form of macular dystrophy. Research indicates that this condition is transmitted as an autosomal recessive trait in the ABCA4 gene (also known as the ABCR gene). This gene is a member of the ABC Super Family of genes that encode for transmembrane proteins involved in the energy dependent transport of a wide spectrum of substances across membranes.

Symptoms of Stargardt Disease include a decrease in central vision and difficulty with dark adaptation, problems that generally worsen with age so that many persons afflicted with Stargardt Disease experience visual loss of 20/100 to 20/400. Persons with Stargardt Disease are generally encouraged to avoid bright light because of the potential over-production of all-trans-retinal.

Methods for diagnosing Stargardt Disease include the observation of an atrophic or “beaten-bronze” appearance of deterioration in the macula, and the presence of numerous yellowish-white spots that occur within the retina surrounding the atrophic-appearing central macular lesion. Other diagnostic tests include the use of an electroretinogram, electrooculogram, and dark adaptation testing. In addition, a fluorescein angiogram can be used to confirm the diagnosis. In this latter test, observation of a “dark” or “silent” choroid appears associated with the accumulation of lipofuscin in the retinal pigment epithelium of the patient, one of the early symptoms of macular degeneration.

Currently, treatment options for the macular degenerations and macular dystrophies are limited. Some patients with dry form AMD have responded to high doses of vitamins and minerals. In addition, a few studies have indicated that laser photocoagulation of drusen prevents or delays the development of drusen that can lead to the more severe symptoms of dry form AMD. Finally, certain studies have shown that extracorporeal rheopheresis benefits patients with dry form AMD.

However, successes have been limited and there continues to be a strong desire for new methods and treatments to manage and limit vision loss associated with the macular degenerations and dystrophies.

### SUMMARY OF THE INVENTION

Presented herein are methods, compositions and formulations for (a) treating ophthalmic conditions, and (b) controlling symptoms that presage (e.g., risk factors) or are associated with such ophthalmic conditions, wherein the compositions and formulations do not directly inhibit or antagonize any of the visual cycle proteins at the concentrations used to treat ophthalmic conditions, or control symptoms that presage (e.g., risk factors) or are associated with such ophthalmic conditions. In one aspect, such methods and formulations comprise the use of retinyl derivatives. In further aspects, such methods and formulations comprise the use of agents to treat ophthalmic conditions by lowering the level of serum retinol, serum retinol binding protein (RBP), and/or serum retinol-RBP in the body of a patient. In further aspects the ophthalmic conditions are retinopathies. In further aspects the ophthalmic conditions are lipofuscin-based retinal diseases. In further aspects, the lipofuscin-based retinal diseases are macular degenerations, macular dystrophies and retinal dystrophies. In further aspects, the methods and formulations are used to protect eyes of a mammal from light; in other aspects the methods and formulations are used to limit the formation of all-trans-retinal, N-retinylidene-N-retinylethanolamine, N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, N-retinylidene-phosphatidylethanolamine, lipofuscin, geographic atrophy, scotoma, photoreceptor degeneration and/or drusen in the eye of a mammal. In other aspects, such methods and formulations comprise the use of agents that can cause a reduction of rod-dominated maximum ERG a-wave amplitude in a patient. In yet other aspects, the methods and formulations are used in combination with other treatment modalities.

In another aspect are methods for treating a lipofuscin-based retinal disease comprising modulating the serum level of retinol, RBP, and/or retinol-RBP in the body of a mammal, including embodiments wherein (a) the lipofuscin-based retinal disease is juvenile macular degeneration, including Stargardt Disease; (b) the lipofuscin-based retinal disease is dry form age-related macular degeneration; (c) the lipofuscin-based retinal disease is cone-rod dystrophy; (d) the lipofuscin-based retinal disease is retinitis pigmentosa; (e) the lipofuscin-based retinal disease is wet-form age-related macular degeneration; (f) the lipofuscin-based retinal disease is or presents geographic atrophy and/or photoreceptor degeneration; or (g) the lipofuscin-based retinal disease is a lipofuscin-based retinal degeneration.

In another aspect are methods for treating a lipofusin-based retinal disease in a mammal comprising reducing the serum retinol, serum retinol binding protein (RBP), and/or serum retinol-RBP level in the mammal by a desired percentage. In certain embodiments, the desired percentage of serum retinol, serum retinol binding protein (RBP), and/or serum retinol-RBP reduction is relative to pre-therapeutic levels; in alternative embodiments, the desired percentage of serum retinol, serum retinol binding protein (RBP), and/or serum retinol-RBP reduction is relative to a pre-determined threshold level. In certain embodiments, the desired percentage of serum retinol, serum retinol binding protein (RBP), and/or serum retinol-RBP reduction is at least about 10%, at least about 20%, at least about 30%, at least about 40%, at least about 50%, at least about 60%, at least about 70%, or at least about 80%. In certain embodiments, the desired percentage of serum retinol, serum retinol binding protein (RBP), and/or serum retinol-RBP reduction is no more than about 30%, no more than about 40%, no more than about 50%, no more than about 60%, no more than about 70%, no more than about 80%, no more than about 85%, no more than about 90%, or no more than about 95%. In certain embodiments, the desired percentage of serum retinol, serum retinol binding protein (RBP), and/or serum retinol-RBP reduction is between about 20 and about 75% of the pre-treatment baseline value. In certain embodiments, the desired percentage of serum retinol, serum retinol binding protein (RBP), and/or serum retinol-RBP reduction is maintained for at least 1 week, for at least 1 month, for at least 6 months, for at least 1 year, for the lifetime of the mammal.

In another aspect are methods for treating a lipofusin-based retinal disease in a mammal comprising maintaining the serum retinol, serum retinol binding protein (RBP), and/or serum retinol-RBP level in the mammal within a desired range. In certain embodiments, the desired range of serum retinol, serum retinol binding protein (RBP), and/or serum retinol-RBP is greater than a level that leads to diseases or conditions associated with Vitamin A deficiency and less than a level that increases the accumulation of A2E in at least one eye of the mammal. In certain embodiments, the level of serum retinol, serum retinol binding protein (RBP), and/or serum retinol-RBP that increases the accumulation of A2E in at least one eye of the mammal is at least about 10%, at least about 20%, at least about 30%, at least about 40%, at least about 50%, at least about 60%, at least about 70%, or at least about 80% of the pre-therapy serum retinol, serum retinol binding protein (RBP), and/or serum retinol-RBP level. In certain embodiments, the level of serum retinol, serum retinol binding protein (RBP), and/or serum retinol-RBP that leads to diseases or conditions associated with Vitamin A deficiency is no more than about 30%, no more than about 40%, no more than about 50%, no more than about 60%, no more than about 70%, no more than about 80%, no more than about 85%, no more than about 90%, or no more than about 95% of the pre-therapy serum retinol, serum retinol binding protein (RBP), and/or serum retinol-RBP level. In certain embodiments, the desired percentage of serum retinol, serum retinol binding protein (RBP), and/or serum retinol-RBP reduction is between about 20% and about 75% of the pre-treatment baseline value. In certain embodiments, the desired percentage of serum retinol, serum retinol binding protein (RBP), and/or serum retinol-RBP reduction is maintained for at least 1 week, for at least 1 month, for at least 6 months, for at least 1 year, for the lifetime of the mammal. In certain embodiments, the serum retinol, serum retinol binding protein (RBP), and/or serum retinol-RBP level in the mammal is measured at periodic levels to make sure that the serum retinol, serum retinol binding protein (RBP), and/or serum retinol-RBP level is maintained within a desired range.

In another aspect are methods for treating a lipofusin-based retinal disease in a mammal comprising reducing the retinol level in at least one RPE of the mammal by a desired percentage. In certain embodiments, the desired percentage of retinol reduction is relative to pre-therapeutic levels; in alternative embodiments, the desired percentage of retinol reduction is relative to a pre-determined threshold level. In certain embodiments, the desired percentage of retinol reduction is at least about 10%, at least about 20%, at least about 30%, at least about 40%, at least about 50%, at least about 60%, at least about 70%, or at least about 80%. In certain embodiments, the desired percentage of retinol reduction is no more than about 30%, no more than about 40%, no more than about 50%, no more than about 60%, no more than about 70%, no more than about 80%, no more than about 85%, no more than about 90%, or no more than about 95%. In certain embodiments, the desired percentage of RPE retinol reduction is between about 20% and about 75% of the pre-treatment baseline value. In certain embodiments, the desired percentage of retinol reduction is maintained for at least 1 week, for at least 1 month, for at least 6 months, for at least 1 year, for the lifetime of the mammal.

The level of serum retinol, serum RBP, and serum retinol-RBP are inter-related. Reduction of the level of any one of these biological materials will lead to a reduction in the levels of the other two biological materials. Thus, hereinafter, the term “serum retinol” refers to any one or all of serum retinol, serum RBP, and serum retinol-RBP.

In a further aspect the serum retinol levels in the body of the mammal are modulated by methods comprising administering to the mammal at least once an effective amount of a first compound having the structure of Formula (I):

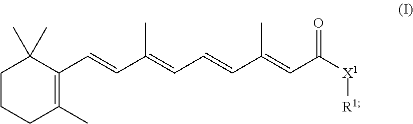

wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle, provided that R³ is not H when both x is 0 and L¹ is a single bond; or an active metabolite, or a pharmaceutically acceptable prodrug or solvate thereof.

In a further aspect are methods for reducing the level of all-trans retinal in an eye of a mammal comprising modulating the serum retinol level in the mammal by administering to the mammal at least once an effective amount of a first compound having the structure of Formula (I).

In another aspect are methods for reducing the formation of N-retinylidene-N-retinylethanolamine, N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine, in an eye of a mammal comprising modulating the serum retinol level in the mammal by administering to the mammal at least once an effective amount of a first compound having the structure of Formula (I).

In another aspect are methods for reducing the formation of lipofuscin in an eye of a mammal comprising modulating the serum retinol level in the mammal by administering to the mammal at least once an effective amount of a first compound having the structure of Formula (I).

In another aspect are methods for reducing the formation of drusen in an eye of a mammal comprising modulating the serum retinol level in the mammal by administering to the mammal at least once an effective amount of a first compound having the structure of Formula (I).

In another aspect are methods for reducing and/or inhibiting choroidal neovascularization in the eye of a mammal comprising modulating the serum retinol levels in the mammal by administering to the mammal at least once an effective amount of a first compound having the structure of Formula (I). In a further embodiment, the compound is an anti-angiogenic agent.

In another aspect are methods for treating macular degeneration in an eye of a mammal comprising modulating the serum retinol level in the mammal by administering to the mammal at least once an effective amount of a first compound having the structure of Formula (I). In a further embodiment of this aspect, the macular degeneration is juvenile macular degeneration, including Stargardt Disease. In a further embodiment of this aspect, (a) the macular degeneration is dry form age-related macular degeneration, or (b) the macular degeneration is cone-rod dystrophy. In a further embodiment of this aspect, the macular degeneration is the wet form of age-related macular degeneration. In a further embodiment of this aspect, the macular degeneration is choroidal neovascularization, subretinal neovascularization, exudative, or disciform degeneration.

In another aspect are methods for reducing the formation or limiting the spread of geographic atrophy, scotoma, and/or photoreceptor degeneration in an eye of a mammal comprising modulating the serum retinol level in the mammal by administering to the mammal at least once an effective amount of a first compound having the structure of Formula (I).

In another aspect are methods for reducing the formation of abnormal blood vessel growth beneath the macula in an eye of a mammal comprising modulating the serum retinol level in the mammal by administering to the mammal at least once an effective amount of a first compound having the structure of Formula (I).

In another aspect are methods for protecting the photoreceptors in any eye of a mammal comprising modulating the serum retinol level in the mammal by administering to the mammal at least once an effective amount of a first compound having the structure of Formula (I).

In another aspect are methods for protecting an eye of a mammal from light comprising modulating the serum retinol level in the mammal by administering to the mammal at least once an effective amount of a first compound having the structure of Formula (I).

In another aspect is the use of a compound of Formula (I) in the manufacture of a medicament for treating an ophthalmic disease or condition in an animal in which the activity of at least one visual cycle protein contributes to the pathology and/or symptoms of the disease or condition. In one embodiment of this aspect, the visual cycle protein is selected from the group consisting of lecithin-retinol acyltransferase, RPE65, dehydrogenases, isomerases, and cellular retinaldehyde binding protein. In another or further embodiment of this aspect, the ophthalmic disease or condition is a retinopathy. In a further or alternative embodiment, the ophthalmic disease or condition is a lipofuscin-based retinal disease. In a further or alternative embodiment, the lipofuscin-based retinal disease is a macular degeneration. In a further or alternative embodiment, the symptom of the disease or condition is formation of all-trans-retinal, N-retinylidene-N-retinylethanolamine, N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, N-retinylidene-phosphatidylethanolamine, lipofuscin, photoreceptor degeneration, geographic atrophy, scotoma, choroidal neovascularization, and/or drusen in the eye of a mammal.

In any of the aforementioned aspects are further embodiments in which (a) X¹ is NR², wherein R² is H or (C₁-C₄)alkyl; (b) wherein x is 0; (c) x is 1 and L¹ is —C(O)—; (d) R³ is an optionally substituted aryl; (e) R³ is an optionally substituted heteroaryl; (f) X¹ is NH and R³ is an optionally substituted aryl, including yet further embodiments in which (i) the aryl group has one substituent, (ii) the aryl group has one substituent selected from the group consisting of halogen, OH, O(C₁-C₄)alkyl, NH(C₁-C₄)alkyl, O(C₁-C₄)fluoroalkyl, and N[(C₁-C₄)alkyl]₂, (iii) the aryl group has one substituent, which is OH, (v) the aryl is a phenyl, or (vi) the aryl is naphthyl; (g) the compound

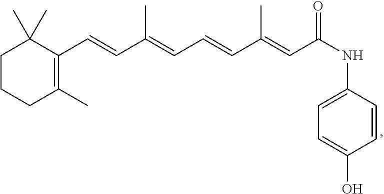

is or an active metabolite, or a pharmaceutically acceptable prodrug or solvate thereof; (h) the compound is 4-hydroxyphenylretinamide, or a metabolite, or a pharmaceutically acceptable prodrug or solvate thereof; (i) the compound is 4-methoxyphenylretinamide, or (j) 4-oxo fenretinide, or a metabolite, or a pharmaceutically acceptable prodrug or solvate thereof.

In any of the aforementioned aspects are embodiments wherein a measured level of serum retinol that is greater than a level associated with an increase in the accumulation of A2E in at least one eye of the mammal is an indication that the next dose of a compound having the structure of Formula (I) should be increased. In certain embodiments, a measured level of serum retinol that is less than a level associated with Vitamin A deficiency is an indication that the next dose of a compound having the structure of Formula (I) should be decreased. In either of these embodiments, the health of the mammal and the level of A2E accumulation are additional factors that can be considered prior to adjusting the subsequent dose of a compound having the structure of Formula (I).

In any of the aforementioned aspects, the amount of compound used to lower the serum retinol level in the mammal is not sufficient to inhibit the regeneration of visual chromophore in the mammal.

In any of the aforementioned aspects are further embodiments in which (a) the effective amount of the compound is systemically administered to the mammal; (b) the effective amount of the compound is administered orally to the mammal; (c) the effective amount of the compound is intravenously administered to the mammal; or (d) the effective amount of the compound is administered by injection to the mammal.

In any of the aforementioned aspects are further embodiments in which the mammal is a human, including embodiments wherein (a) the human is a carrier of the mutant ABCA4 gene for Stargardt Disease or the human has a mutant ELOV4 gene for Stargardt Disease, or has a genetic variation in complement factor H associated with age-related macular degeneration, or (b) the human has an ophthalmic condition or trait selected from the group consisting of Stargardt Disease, recessive retinitis pigmentosa, geographic atrophy, scotoma, photoreceptor degeneration, dry-form AMD, recessive cone-rod dystrophy, exudative age-related macular degeneration, cone-rod dystrophy, and retinitis pigmentosa. In any of the aforementioned aspects are further embodiments in which the mammal is an animal model for retinal degeneration, examples of which are provided herein.

In any of the aforementioned aspects are further embodiments comprising multiple administrations of the effective amount of the compound, including further embodiments in which (i) the time between multiple administrations is at least one week; (ii) the time between multiple administrations is at least one day; and (iii) the compound is administered to the mammal on a daily basis; or (iv) the compound is administered to the mammal every 12 hours.

In any of the aforementioned aspects are further embodiments comprising administering at least one additional agent selected from the group consisting of an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, resveratrol and other trans-stilbene compounds, an agent that inhibits, antagonizes or short-circuits the visual cycle at a step of the visual cycle that occurs outside a disc of a rod photoreceptor cell, and an agent that reduces serum retinol levels. In further embodiments:
(a) the additional agent is an inducer of nitric oxide production, including embodiments in which the inducer of nitric oxide production is selected from the group consisting of citrulline, ornithine, nitrosated L-arginine, nitrosylated L-arginine, nitrosated N-hydroxy-L-arginine, nitrosylated N-hydroxy-L-arginine, nitrosated L-homoarginine and nitrosylated L-homoarginine;
(b) the additional agent is an anti-inflammatory agent, including embodiments in which the anti-inflammatory agent is selected from the group consisting of a non-steroidal anti-inflammatory drug, a lipoxygenase inhibitor, prednisone, dexamethasone, and a cyclooxygenase inhibitor;
(c) the additional agent is at least one physiologically acceptable antioxidant, including embodiments in which the physiologically acceptable antioxidant is selected from the group consisting of Vitamin C, Vitamin E, beta-carotene, Coenzyme Q, and 4-hydroxy-2,2,6,6-tetramethylpiperadine-N-oxyl, or embodiments in which (i) the at least one physiologically acceptable antioxidant is administered with the compound having the structure of Formula (I), or (ii) at least two physiologically acceptable antioxidants are administered with the compound having the structure of Formula (I);
(d) the additional agent is at least one physiologically acceptable mineral, including embodiments in which the physiologically acceptable mineral is selected from the group consisting of a zinc (II) compound, a Cu(II) compound, and a selenium (II) compound, or embodiments further comprising administering to the mammal at least one physiologically acceptable antioxidant;
(e) the additional agent is a negatively charged phospholipid, including embodiments in which the negatively charged phospholipid is phosphatidylglycerol;
(f) the additional agent is a carotenoid, including embodiments in which the carotenoid is selected from the group consisting of lutein, astaxanthin and zeaxanthin;
(g) the additional agent is a statin, including embodiments in which the statin is selected from the group consisting of rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium, and dihydrocompactin;
(h) the additional agent is an anti-angiogenic drug, including embodiments in which the anti-angiogenic drug is Rhufab V2, Tryptophanyl-tRNA synthetase, an Anti-VEGF pegylated aptamer, Squalamine, anecortave acetate, Combretastatin A4 Prodrug, Macugen™, mifepristone, subtenon triamcinolone acetonide, intravitreal crystalline triamcinolone acetonide, AG3340, fluocinolone acetonide, and VEGF-Trap;
(i) the additional agent is a matrix metalloproteinase inhibitor, including embodiments in which the matrix metalloproteinase inhibitor is a tissue inhibitors of metalloproteinases, α₂-macroglobulin, a tetracycline, a hydroxamate, a chelator, a synthetic MMP fragment, a succinyl mercaptopurine, a phosphonamidate, and a hydroxaminic acid;
(j) the additional agent is an agent that inhibits, antagonizes or short-circuits the visual cycle at a step of the visual cycle that occurs outside a disc of a rod photoreceptor cell, including 13-cis-retinoic acid, all-trans-retinoic acid, or any agent disclosed in paragraphs 111-765 of U.S. Patent Application Publication No. 20060069078 (the contents of which are incorporated by reference);
(k) the additional agent is resveratrol or other trans-stilbene compounds;
(l) the additional agent reduces the serum retinol level in a mammal;
(m) the additional agent is administered (i) prior to the administration of the compound having the structure of Formula (I), (ii) subsequent to the administration of the compound having the structure of Formula (I), (iii) simultaneously with the administration of the compound having the structure of Formula (I), or (iv) both prior and subsequent to the administration of the compound having the structure of Formula (I); or
(n) the additional agent and the compound having the structure of Formula (I), are administered in the same pharmaceutical composition.

In any of the aforementioned aspects are further embodiments comprising administering extracorporeal rheopheresis to the mammal.

In any of the aforementioned aspects are further embodiments comprising reducing the amount of Vitamin A in the diet of the mammal.

In any of the aforementioned aspects are further embodiments comprising administering to the mammal a therapy selected from the group consisting of limited retinal translocation, photodynamic therapy, drusen lasering, macular hole surgery, macular translocation surgery, Phi-Motion, Proton Beam Therapy, Retinal Detachment and Vitreous Surgery, Scleral Buckle, Submacular Surgery, Transpupillary Thermotherapy, Photosystem I therapy, MicroCurrent Stimulation, anti-inflammatory agents, RNA interference, administration of eye medications such as phospholine iodide or echothiophate or carbonic anhydrase inhibitors, microchip implantation, stem cell therapy, gene replacement therapy, ribozyme gene therapy, photoreceptor/retinal cells transplantation, and acupuncture.

In any of the aforementioned aspects are further embodiments comprising the use of laser photocoagulation to remove drusen from the eye of the mammal.

In any of the aforementioned aspects are further embodiments comprising administering to the mammal at least once an effective amount of a second compound having the structure of Formula (I), wherein the first compound is different from the second compound.

In any of the aforementioned aspects are further embodiments comprising (a) monitoring formation of drusen in the eye of the mammal; (b) measuring levels of lipofuscin in the eye of the mammal by autofluorescence; (c) measuring visual acuity in the eye of the mammal; (d) conducting a visual field examination on the eye of the mammal, including embodiments in which the visual field examination is a Humphrey visual field exam and/or microperimetry; (e) measuring the autofluorescence or absorption spectra of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine in the eye of the mammal; (f) conducting a reading speed and/or reading acuity examination; (g) measuring scotoma size; or (h) measuring the size and number of the geographic atrophy lesions.

In any of the aforementioned aspects are further embodiments comprising determining whether the mammal is a carrier of the mutant ABCA4 allele for Stargardt Disease or has a mutant ELOV4 allele for Stargardt Disease or has a genetic variation in complement factor H associated with age-related macular degeneration.

In any of the aforementioned aspects are further embodiments comprising an additional treatment for retinal degeneration.

In another aspect are pharmaceutical compositions comprising an effective amount of compound having the structure:

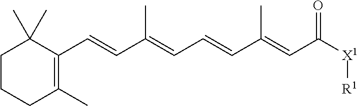

wherein X₁ is selected from the group consisting of NR², O, S, CHR²; R¹ is (CHR²)ₓ-L¹-R³, wherein x is 0, 1, 2, or 3; L¹ is a single bond or —C(O)—; R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and R³ is H or a moiety, optionally substituted with 1-3 independently selected substituents, selected from the group consisting of (C₂-C₇)alkenyl, (C₂-C₇)alkynyl, aryl, (C₃-C₇)cycloalkyl, (C₅-C₇)cycloalkenyl, and a heterocycle; provided that R is not H when both x is 0 and L¹ is a single bond; or an active metabolite, or a pharmaceutically acceptable prodrug or solvate thereof; and a pharmaceutically acceptable carrier.

In further embodiment of the pharmaceutical composition aspect, (a) the pharmaceutically acceptable carrier comprises lysophosphatidylcholine, monoglyceride and a fatty acid; (b) the pharmaceutically acceptable carrier further comprises flour, a sweetener, and a humectant; (c) the pharmaceutically acceptable carrier comprises corn oil and a non-ionic surfactant; (d) the pharmaceutically acceptable carrier comprises dimyristoyl phosphatidylcholine, soybean oil, t-butyl alcohol and water; (e) the pharmaceutically acceptable carrier comprises ethanol, alkoxylated caster oil, and a non-ionic surfactant; (f) the pharmaceutically acceptable carrier comprises an extended release formulation; or (g) the pharmaceutically acceptable carrier comprises a rapid release formulation.

In further embodiment of the pharmaceutical composition aspect, the pharmaceutical composition further comprising an effective amount of at least one additional agent selected from the group consisting of an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, resveratrol and other trans-stilbene compounds, and an agent that inhibits, antagonizes or short-circuits the visual cycle at a step of the visual cycle that occurs outside a disc of a rod photoreceptor cell, including 13-cis-retinoic acid, all-trans-retinoic acid, or any agent disclosed in paragraphs 111-765 of U.S. Patent Application Publication No. 20060069078 (the contents of which are incorporated by reference). In further embodiments, (a) the additional agent is a physiologically acceptable antioxidant; (b) the additional agent is an inducer of nitric oxide production; (c) the additional agent is an anti-inflammatory agent; (d) the additional agent is a physiologically acceptable mineral; (e) the additional agent is a negatively charged phospholipid; (f) the additional agent is a carotenoid; (g) the additional agent is a statin; (h) the additional agent is an anti-angiogenic agent; (i) the additional agent is a matrix metalloproteinase inhibitor; (j) the additional agent is an agent that inhibits, antagonizes or short-circuits the visual cycle at a step of the visual cycle that occurs outside a disc of a rod photoreceptor cell, including 13-cis-retinoic acid, all-trans-retinoic acid, or any agent disclosed in paragraphs 111-765 of U.S. Patent Application Publication No. 20060069078 (the contents of which are incorporated by reference); or (k) resveratrol and other trans-stilbene compounds.

Also described herein are methods and compositions for treating a patient with retinal-related diseases by modulating RBP or TTR levels in the patient by administration of at least one modulating compound. In a further embodiment the retinol-related diseases are lipofuscin-based retinal diseases. In a further embodiment the modulation of RBP and/or TTR levels in the patient provide a reduction in serum retinol levels in the patient. In a further embodiment, the reduction of serum retinol levels in the patient results in the reduction of retinoids in at least one eye of the patient. In a further embodiment, the reduction of serum retinol levels in the patient results in the reduction of the A2E level in at least one eye of the patient. In a further embodiment, the modulating compound has the structure of Formula (I). In a further embodiment, the modulating compound is fenretinide or an active metabolite thereof. In a further embodiment, the modulating compound does not have the structure of Formula (I), but is selected from the modulating compounds described herein and by using the screening methods described herein.

In one embodiment, the methods and compositions disclosed herein provide for modulating RBP or TTR levels in a mammal comprising administering to the mammal at least once an effective amount of an agent which modulates RBP binding to TTR in said mammal, wherein said modulation of RBP or TTR levels reduces the formation of all-trans retinal in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

The methods and compositions disclosed herein also provide for modulating RBP or TTR levels in a mammal comprising administering to the mammal at least once an effective amount of an agent which increases the clearance rate of RBP or TTR in said mammal, wherein said modulation of RBP or TTR levels reduces the formation of all-trans retinal in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In one embodiment, the methods and compositions disclosed herein provide for modulating RBP or TTR levels in a mammal comprising administering to the mammal at least once an effective amount of an agent which modulates RBP binding to TTR in said mammal, wherein said modulation of RBP or TTR levels reduces the formation of N-retinylidene-N-retinylethanolamine in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In yet another embodiment, the methods and compositions disclosed herein provide for modulating RBP or TTR levels in a mammal comprising administering to the mammal at least once an effective amount of an agent which increases the clearance rate of RBP or TTR in said mammal, wherein said modulation of RBP or TTR levels reduces the formation of N-retinylidene-N-retinylethanolamine in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In yet another embodiment, the methods and compositions disclosed herein provide for modulating RBP or TTR levels in a mammal comprising administering to the mammal at least once an effective amount of an agent which increases the clearance rate of RBP or TTR in said mammal, wherein said modulation of RBP or TTR levels reduces the formation of lipofuscin in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In one embodiment, the methods and compositions disclosed herein provide for modulating RBP or TTR levels in a mammal comprising administering to the mammal at least once an effective amount of an agent which modulates RBP binding to TTR in said mammal, wherein said modulation of RBP or TTR levels reduces the formation of drusen in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In another embodiment, the methods and compositions disclosed herein provide for modulating RBP or TTR levels in a mammal comprising administering to the mammal at least once an effective amount of an agent which increases the clearance rate of RBP or TTR in said mammal, wherein said modulation of RBP or TTR levels reduces the formation of drusen in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In yet another embodiment, the methods and compositions disclosed herein provide for modulating RBP or TTR levels in a mammal comprising administering to the mammal at least once an effective amount of an agent which modulates RBP binding to TTR in said mammal, wherein said modulation of RBP or TTR levels modulates lecithin-retinol acyltransferase in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In another embodiment, the methods and compositions disclosed herein provide for modulating RBP or TTR levels in a mammal comprising administering to the mammal at least once an effective amount of an agent which increases the clearance rate of RBP or TTR in said mammal, wherein said modulation of RBP or TTR levels modulates lecithin-retinol acyltransferase in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In yet another embodiment, the methods and compositions disclosed herein provide for modulating RBP or TTR levels in a mammal comprising administering to the mammal at least once an effective amount of an agent which modulates RBP binding to TTR in said mammal, wherein said modulation of RBP or TTR levels prevents age-related macular degeneration or dystrophy in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

The methods and compositions disclosed herein also provide for modulating RBP or TTR levels in a mammal comprising administering to the mammal at least once an effective amount of an agent which increases the clearance rate of RBP or TTR in said mammal, wherein said modulation of RBP or TTR levels prevents age-related macular degeneration or dystrophy in an eye of a mammal. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In yet another embodiment, the methods and compositions disclosed herein provide for modulating RBP or TTR levels in a mammal comprising administering to the mammal at least once an effective amount of an agent which modulates RBP binding to TTR in said mammal, wherein said modulation of RBP or TTR levels protects an eye of a mammal from light. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In another embodiment, the methods and compositions disclosed herein provide for modulating RBP or TTR levels in a mammal comprising administering to the mammal at least once an effective amount of an agent which increases the clearance rate of RBP or TTR in said mammal, wherein said modulation of RBP or TTR levels protects an eye of a mammal from light. In one embodiment, the agent is chosen from the compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

In one embodiment, the methods and compositions disclosed herein provide for modulating retinol binding protein (RBP) or transthyretin (TTR) levels in a mammal comprising administering to the mammal at least once an effective amount of at least one of the compounds chosen from the group consisting of an an RBP clearance agent, a TTR clearance agent, an RBP antagonist, an RBP agonist, a TTR antagonist and a TTR agonist.

In another embodiment, the RBP clearance agent is chosen from compounds having the structure of Formula (I). In a further embodiment, the compound is fenretinide or an active metabolite thereof. In another embodiment, the RBP agonist or antagonist is chosen from compounds having the structure of Formula (I). In a further embodiment, the compound is fenretinide or an active metabolite thereof. In a further embodiment, the compound does not have the structure of Formula (I), but is selected from the modulating compounds described herein and by using the screening methods described herein.

The methods and compositions disclosed herein also provide for the treatment of age-related macular degeneration or dystrophy, comprising administering to a mammal at least once an effective amount of a first compound, wherein said first compound modulates RBP or TTR levels in the mammal. In one embodiment, the first compound increases RBP or TTR clearance in the mammal. In still another embodiment, the first compound inhibits RBP binding to TTR.

The methods and compositions disclosed herein also provide for the reduction of formation of all-trans retinal in an eye of a mammal comprising administering to the mammal at least once an effective amount of a first compound, wherein the first compound modulates RBP or TTR levels in the mammal. In one embodiment, the first compound increases RBP or TTR clearance in the mammal. In still another embodiment, the first compound inhibits RBP binding to TTR.

In one embodiment, the methods and compositions disclosed herein provide for reducing the formation of N-retinylidene-N-retinylethanolamine in an eye of a mammal comprising administering to the mammal at least once an effective amount of a first compound, wherein said first compound modulates RBP or TTR levels in the mammal. In one embodiment, the first compound increases RBP or TTR clearance in the mammal. In still another embodiment, the first compound inhibits RBP binding to TTR.

In yet another embodiment, the methods and compositions disclosed herein provide for reducing the formation of lipofuscin in an eye of a mammal comprising administering to the mammal at least once an effective amount of a first compound, wherein said first compound modulates RBP or TTR levels in the mammal. In one embodiment, the first compound increases RBP or TTR clearance in the mammal. In still another embodiment, the first compound inhibits RBP binding to TTR.

In another embodiment, the methods and compositions disclosed herein provide for reducing the formation of drusen in an eye of a mammal comprising administering to the mammal at least once an effective amount of a first compound, wherein said first compound modulates RBP or TTR levels in the mammal. In one embodiment, the first compound increases RBP or TTR clearance in the mammal. In still another embodiment, the first compound inhibits RBP binding to TTR.

In one embodiment, the methods and compositions disclosed herein provide for protecting an eye of a mammal from light comprising administering to the mammal at least once an effective amount of a first compound, wherein said first compound modulates RBP or TTR levels in the mammal. In one embodiment, the first compound increases RBP or TTR clearance in the mammal. In still another embodiment, the first compound inhibits RBP binding to TTR.

In another embodiment, the methods and compositions disclosed herein provide for the treatment of retinol-related diseases, comprising administering to the mammal at least once an effective amount of at least one of the compounds chosen from the group consisting of: an RBP clearance agent, a TTR clearance agent, an RBP antagonist, an RBP agonist, a TTR antagonist, a TTR agonist and a retinol binding receptor antagonist.

In one embodiment, the RBP clearance agent is chosen from compounds having the structure of Formula (I). In a further embodiment, the compound is fenretinide or an active metabolite thereof. In yet another embodiment, the TTR clearance agent is chosen from compounds having the structure of Formula (I). In a further embodiment, the compound is fenretinide or an active metabolite thereof. In yet another embodiment, the RBP agonist or antagonist is chosen from compounds having the structure of Formula (I). In a further embodiment, the compound is fenretinide or an active metabolite thereof.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All references cited herein, including patents, patent applications, and publications, are hereby incorporated by reference in their entirety.

### BRIEF DESCRIPTION OF THE FIGURES

FIGS. 1a-1c illustrate various reverse phase LC analyses of acetonitrile extracts of serum. The serum was obtained from mice administered with either DMSO (FIG. 1a), 10 mg/kg N-4-(hydroxyphenyl)retinamide (HPR) (FIG. 1b), or 20 mg/kg HPR (FIG. 1c) for 14 days.

FIG. 2a illustrates ocular concentrations of all-trans retinol (atROL) and HPR as a function of time in mice following injection of 10 mg/kg HPR.

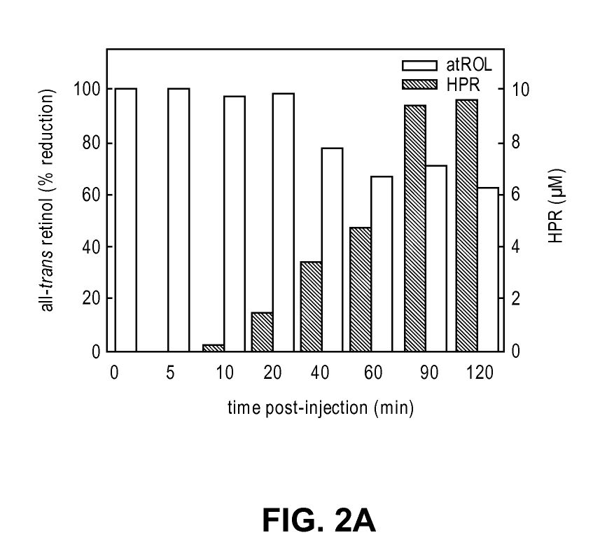

FIG. 2b illustrates serum concentrations of all-trans retinol and HPR in mice following 14-day treatment with DMSO, 10 mg/kg HPR, or 20 mg/kg HPR; see FIG. 7 for an updated and corrected version of this figure.

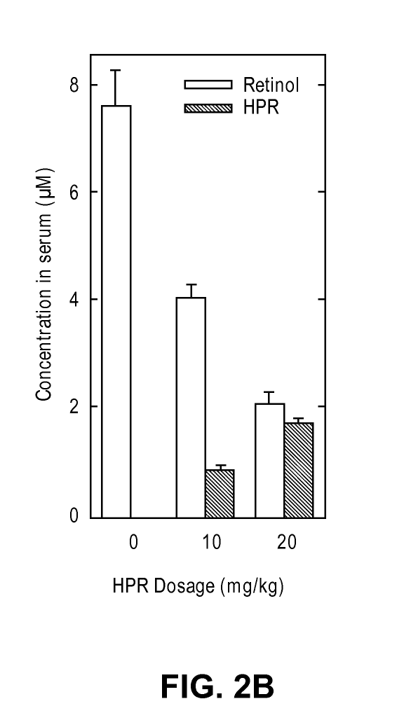

FIG. 3a illustrates a control binding assay for the interaction between retinol and retinol-binding protein as measured by fluorescence quenching.

FIG. 3b illustrates a binding assay for the interaction between retinol and retinol-binding protein in the presence of HPR (2 μM) as measured by fluorescence quenching.

FIG. 4a illustrates the effect of HPR on A2PE-H₂ biosynthesis in abca4 null mutant mice.

FIG. 4b illustrates the effect of HPR on A2E biosynthesis in abca4 null mutant mice.

FIG. 5 illustrates the modulation of Retinol Binding Protein (RBP) binding to Transthyretin (TTR) by N-4-(methoxyphenyl)retinamide (MPR) as measured by fluorescence quenching.

FIG. 6 illustrates the modulation of RBP binding to TTR by MPR as measured by size exclusion chromatography and UV/Visible spectrophotometry.

FIG. 7 illustrates the analysis of serum retinol as a function of fenretinide concentration.

FIG. 8 illustrates a correlation plot relating fenretinide concentration to reductions in retinol, A2PE-H₂ and A2E in ABCA4 null mutant mice.

FIG. 9 illustrates retinoid composition in light adapted DMSO- and HPR-treated mice (panel A); the affect of HPR on the regeneration of visual chromophore (panel B); the effect of HPR on bleached chromophore recycling (panel C); and electrophysiological measurements of rod function (panel D), rod and cone function (panel E), and recovery from photobleaching (panel F).

FIG. 10 illustrates the analysis of A2PE-H₂ levels as a function of fenretinide dose and treatment period (panels A-F) and lipofuscin autofluorescence in the RPE of abcr null mutant mice as a function of treatment (panels G-I).

FIG. 11 illustrates light microscopy images of the retinas from DMSO- and HPR-treated animals.

FIG. 12 illustrates the relationship of serum HPR levels to serum retinol levels and ocular levels of retinoids and A2E.

FIG. 13 illustrates a non-limiting example of the binding of retinol and HPR to Retinol Binding Protein.

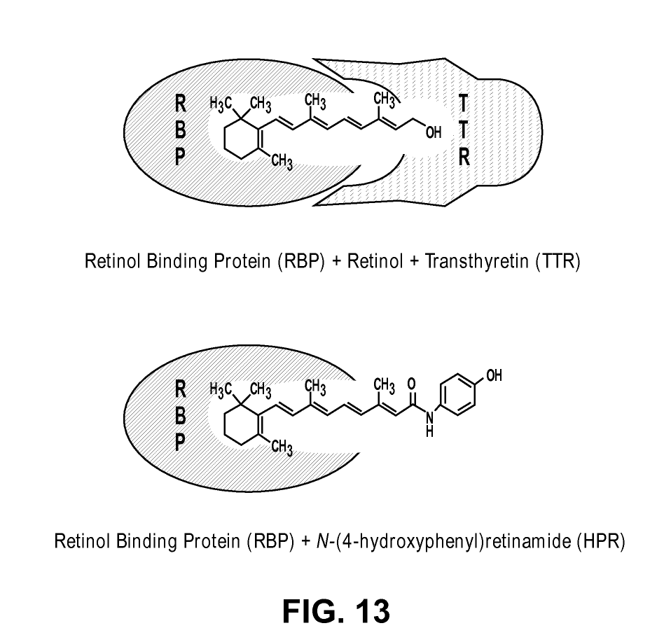

FIG. 14 illustrates the effect of different doses of HPR on the accumulation of retinoid in the eye.

FIG. 15 illustrates the effect of HPR on the levels of 11-cis-retinal and all-trans-retinal in dark adapted and light-adapted abca-4−/− mice.

FIG. 16 illustrates steady-state retinoid levels and rates of visual chromophore regeneration evaluated in abca-4−/− mice following a 28-day treatment period with 10 mg/kg HPR.

FIG. 17 illustrates the delay in the time required to regain dark sensitivity in wild-type and abca-4−/− mice treated with 13-cis-retinoic acid and in abca-4−/− mice treated with HPR.

FIG. 18 illustrates the relative concentration of A2E, A2PE and A2PE-H₂ in three lines of mice at three months of age.

### DETAILED DESCRIPTION OF THE INVENTION

Compounds having the structure of Formula (I) have been used for the treatment of cancer. In particular, the compound N-(4-hydroxyphenyl)retinamide, also known as fenretinide, HPR or 4-HPR, has been extensively tested for the treatment of breast cancer. Moon, et al., Cancer Res., 39:1339-46 (1979). Fenretinide is described in U.S. Pat. Nos. 4,190,594 and 4,323,581. In addition, other methods for preparing fenretinide are known, and further, numerous analogs of fenretinide have been prepared and tested for their effectiveness in treating cancer. See, e.g., U.S. Patent Application Publication 2004/0102650; U.S. Pat. No. 6,696,606; Villeneuve & Chan, Tetrahedron Letters, 38:6489-92 (1997); Um, S. J., et al., Chem. Pharm. Bull., 52:501-506 (2004). Of concern, however, has been the general tendency of such compounds to produce certain side-effects in human patients, including impairment of night vision. See, e.g., Decensi, A., et al., J. Natl. Cancer Inst., 86:1-5-110 (1994); Mariani, L., Tumori., 82:444-49 (1996). A recent study has also provided some evidence that N-(4-hydroxyphenyl)retinamide can induce neuronal-like differentiation in certain cultured human RPE cells. See Chen, S., et al., J. Neurochem., 84:972-81 (2003).

Surprisingly, the compounds of Formula (I) can be used to provide benefit to patients suffering from or susceptible to various macular degenerations and dystrophies, including but not limited to dry-form age-related macular degeneration and Stargardt Disease. Specifically, compounds of Formula (I) provide at least some of the following benefits to such human patients: reduction in the amount of all-trans-retinal (atRAL), reduction in the formation of A2E, reduction in the formation of lipofuscin, reduction in the formation of drusen, and reduction in light sensitivity. There is a reduced tendency to form A2E in ophthalmic and ocular tissues caused, in part, by a reduction in the over-accumulation of all-trans-retinal in these tissues. Because A2E itself is cytotoxic to the RPE (which can lead to retina cell death), administration of compounds having the structure of Formula (I) (alone, or in combination with other agents, as described herein) reduces the rate of accumulation of A2E, a cytotoxic agent, thus providing patient benefit. In addition, because A2E is the major fluorophore of lipofuscin, reduced quantities of A2E in ophthalmic and ocular tissues also results in a reduced tendency to accumulate lipofuscin in such tissues. Thus, in some respects the methods and compositions described herein can be considered to be lipofuscin-based treatments because administration of compounds having the structure of Formula (I) (alone, or in combination with other agents, as described herein) reduces, lowers or otherwise impacts the accumulation of lipofuscin in ophthalmic and/or ocular tissues. A reduction in the rate of accumulation of lipofuscin in ophthalmic and/or ocular tissues benefits patients that have diseases or conditions such as macular degenerations and/or dystrophies.

In addition, because dry-form age-related macular degeneration is often a precursor to wet-form age-related macular degeneration, the use of compounds of Formula (I) can also be used as a preventative therapy for this latter ophthalmic condition. In addition, the compounds of Formula (I) may provide further therapeutic effect for wet-form age-related macular degeneration because such compounds additionally have anti-angiogenic activity.

The Visual Cycle

The vertebrate retina contains two types of photoreceptor cells—rods and cones. Rods are specialized for vision under low light conditions. Cones are less sensitive, provide vision at high temporal and spatial resolutions, and afford color perception. Under daylight conditions, the rod response is saturated and vision is mediated entirely by cones. Both cell types contain a structure called the outer segment comprising a stack of membranous discs. The reactions of visual transduction take place on the surfaces of these discs. The first step in vision is absorption of a photon by an opsin-pigment molecule (rhodopsin), which involves 11-cis to all-trans isomerization of the chromophore. Before light sensitivity can be regained, the resulting all-trans-retinal must be converted back 11-cis-retinal in a multi-enzyme process which takes place in the retinal pigment epithelium, a monolayer of cells adjacent to the retina.

Proper vitamin A homeostasis in the eye relies upon delivery of retinol from serum to the RPE and processing of intracellular vitamin A stores. Upon entry into the retinal pigment epithelium (RPE), retinol is esterifed to a fatty acyl ester (all-trans retinyl ester) by lecithin retinol acyltransferase (LRAT). All-trans retinyl esters are converted to visual chromophore (11-cis retinal) through sequential hydrolysis/isomerization and oxidation by the activities of Rpe65 and an 11-cis-specific retinol dehydrogenase (11 cRDH), respectively. Cellular retinaldehyde binding protein (CRALBP) binds and transports 11-cis retinal to apical processes of the RPE. Following transfer through the interphotoreceptor matrix, 11-cis retinal combines with opsin to form rhodopsin within photoreceptor cells of the retina. Light exposure isomerizes 11-cis retinal to all-trans retinal and initiates a transduction cascade which produces visual stimuli. Reduction of all-trans retinal to all-trans retinol is facilitated by all-trans retinol dehydrogenase (atRDH). All-trans retinol leaves photoreceptor cells and re-enters the visual cycle through apical processes of the RPE.

In addition to the synthesis and re-cycling of visual chromophore, the RPE also plays an important role in maintaining the health of photoreceptor cells of the retina. A critical process in this regard is phagocytosis of diurnally shed photoreceptor outer segment (POS) disc membranes. Approximately 10% of the distal portion of POS discs are shed into and digested by the RPE. Nascent disc membranes, which are continually formed at the connecting cilium between the POS and photoreceptor cell body, replace the shed discs thereby maintaining the length, structure and function of the photoreceptor cell.

Lipofuscin accumulates within RPE cells as a result of incomplete digestion of the retinaldehyde-rich POS debris. The principal toxic fluorophore within ocular lipofuscin is the bis-retinoid compound, N-retinylidene-N-retinylethanolamine (A2E). A2E has been shown to compromise the integrity of RPE cells by a variety of mechanisms which lead ultimately to RPE cell death. Loss of the RPE support role results in death of the overlying retina and finally, loss of vision. Massive levels of lipofuscin and A2E are found in mice and humans harboring mutations in the ABCA4 gene. ABCA4 codes for a photoreceptor-specific protein (ABCR) which removes retinaldehyde-lipid conjugates from photoreceptor outer segments. The pathology resulting from the absence of this protein can be readily observed in electron micrographs of RPE prepared from abca-4−/− mice.

Biochemical analyses of extracts obtained from ocular tissues of abca-4−/− mice established all-trans retinal as the first reactant in the A2E biosynthetic pathway. The light-dependent nature of A2E biosynthesis was demonstrated by raising young abca-4−/− mice in total darkness. This treatment halted the accumulation of A2E and led to the hypothesis that limiting the extent of photobleaching and/or reducing retinal levels in the visual cycle would reduce A2E accumulation.

Macular or Retinal Degenerations and Dystrophies

Macular degeneration (also referred to as retinal degeneration) is a disease of the eye that involves deterioration of the macula, the central portion of the retina. Approximately 85% to 90% of the cases of macular degeneration are the “dry” (atrophic or non-neovascular) type. In dry macular degeneration, the deterioration of the retina is associated with the formation of small yellow deposits, known as drusen, under the macula; in addition, the accumulation of lipofuscin in the RPE leads to photoreceptor degeneration and geographic atrophy. This phenomena leads to a thinning and drying out of the macula. The location and amount of thinning in the retina caused by the drusen directly correlates to the amount of central vision loss. Degeneration of the pigmented layer of the retina and photoreceptors overlying drusen become atrophic and can cause a slow loss of central vision. Ultimately, loss of retinal pigment epithelium and underlying photoreceptor cells results in geographic atrophy. Administration of at least one compound having the structure of Formula (I) to a mammal can reduce the formation of, or limit the spread of, photoreceptor degeneration and/or geographic atrophy in the eye of the mammal. By way of example only, administration of HPR and/or MPR to a mammal, can be used to treat photoreceptor degeneration and/or geographic atrophy in the eye of the mammal.

In “wet” macular degeneration new blood vessels form (i.e., neovascularization) to improve the blood supply to retinal tissue, specifically beneath the macula, a portion of the retina that is responsible for our sharp central vision. The new vessels are easily damaged and sometimes rupture, causing bleeding and injury to the surrounding tissue. Although wet macular degeneration only occurs in about 10 percent of all macular degeneration cases, it accounts for approximately 90% of macular degeneration-related blindness. Neovascularization can lead to rapid loss of vision and eventual scarring of the retinal tissues and bleeding in the eye. This scar tissue and blood produces a dark, distorted area in the vision, often rendering the eye legally blind. Wet macular degeneration usually starts with distortion in the central field of vision. Straight lines become wavy. Many people with macular degeneration also report having blurred vision and blank spots (scotoma) in their visual field. Growth promoting proteins called vascular endothelial growth factor, or VEGF, have been targeted for triggering this abnormal vessel growth in the eye. This discovery has lead to aggressive research of experimental drugs that inhibit or block VEGF. Studies have shown that anti-VEGF agents can be used to block and prevent abnormal blood vessel growth. Such anti-VEGF agents stop or inhibit VEGF stimulation, so there is less growth of blood vessels. Such anti-VEGF agents may also be successful in anti-angiogenesis or blocking VEGF's ability to induce blood vessel growth beneath the retina, as well as blood vessel leakiness. Administration of at least one compound having the structure of Formula (I) to a mammal can reduce the formation of, or limit the spread of, wet-form age-related macular degeneration in the eye of the mammal. By way of example only, administration of HPR and/or MPR to a mammal, can be used to treat wet-form age-related macular degeneration in the eye of the mammal. Similarly, the compounds of Formula (I) (including by way of example only, HPR and/or MPR) can be used to treat choroidal neovascularization and the formation of abnormal blood vessels beneath the macula of the eye of a mammal. Such therapeutic effect can result from a number of effects: lowering of serum retinol and thus ocular retinol levels; anti-angiogenic activity, and/or the quelling of geographic atrophy.

Stargardt Disease is a macular dystrophy that manifests as a recessive form of macular degeneration with an onset during childhood. See e.g., Allikmets et al., Science, 277:1805-07 (1997); Lewis et al., Am. J. Hum. Genet., 64:422-34 (1999); Stone et al., Nature Genetics, 20:328-29 (1998); Allikmets, Am. J. Hum. Gen., 67:793-799 (2000); Klevering, et al, Ophthalmology, 111:546-553 (2004). Stargardt Disease is characterized clinically by progressive loss of central vision and progressive atrophy of the RPE overlying the macula. Mutations in the human ABCA4 gene for Rim Protein (RmP) are responsible for Stargardt Disease. Early in the disease course, patients show delayed dark adaptation but otherwise normal rod function. Histologically, Stargardt Disease is associated with deposition of lipofuscin pigment granules in RPE cells.

Mutations in ABCA4 have also been implicated in recessive retinitis pigmentosa, see, e.g., Cremers et al., Hum. Mol. Genet., 7:355-62 (1998), recessive cone-rod dystrophy, see id., and non-exudative age-related macular degeneration, see e.g., Allikmets et al., Science, 277:1805-07 (1997); Lewis et al., Am. J. Hum. Genet., 64:422-34 (1999), although the prevalence of ABCA4 mutations in AMD is still uncertain. See Stone et al., Nature Genetics, 20:328-29 (1998); Allikmets, Am. J. Hum. Gen., 67:793-799 (2000); Klevering, et al, Ophthalmology, 111:546-553 (2004). Similar to Stargardt Disease, these diseases are associated with delayed rod dark-adaptation. See Steinmetz et al., Brit. J. Ophthalm., 77:549-54 (1993). Lipofuscin deposition in RPE cells is also seen prominently in AMD, see Kliffen et al., Microsc. Res. Tech., 36:106-22 (1997) and some cases of retinitis pigmentosa. See Bergsma et al., Nature, 265:62-67 (1977). In addition, an autosomal dominant form of Stargardt Disease is caused by mutations in the ELOV4 gene. See Karan, et al., Proc. Natl. Acad. Sci. (2005).

In addition, there are several types of macular degenerations that affect children, teenagers or adults that are commonly known as early onset or juvenile macular degeneration. Many of these types are hereditary and are looked upon as macular dystrophies instead of degeneration. Some examples of macular dystrophies include: Cone-Rod Dystrophy, Corneal Dystrophy, Fuch's Dystrophy, Sorsby's Macular Dystrophy, Best Disease, and Juvenile Retinoschisis, as well as Stargardt Disease.

### Chemical Terminology

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 5 carbon atoms. The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

The term “alkylamine” refers to the —N(alkyl)ₓH_{y} group, where x and y are selected from the group x=1, y=1 and x=2, y=0. When x=2, the alkyl groups, taken together, can optionally form a cyclic ring system.

The term “alkenyl” refers to a type of alkyl group in which the first two atoms of the alkyl group form a double bond that is not part of an aromatic group. That is, an alkenyl group begins with the atoms —C(R)═C—R, wherein R refers to the remaining portions of the alkenyl group, which may be the same or different. Non-limiting examples of an alkenyl group include —CH═CH, —C(CH₃)═CH, —CH═CCH₃ and —C(CH₃)═CCH₃. The alkenyl moiety may be branched, straight chain, or cyclic (in which case, it would also be known as a “cycloalkenyl” group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH₃ and —C≡CCH₂CH₃. The “R” portion of the alkynyl moiety may be branched, straight chain, or cyclic.

An “amide” is a chemical moiety with formula —C(O)NHR or —NHC(O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “aromatic” or “aryl” refers to an aromatic group which has at least one ring having a conjugated pi electron system and includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

A “cyano” group refers to a —CN group.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Illustrative examples of cycloalkyl groups include the following moieties:

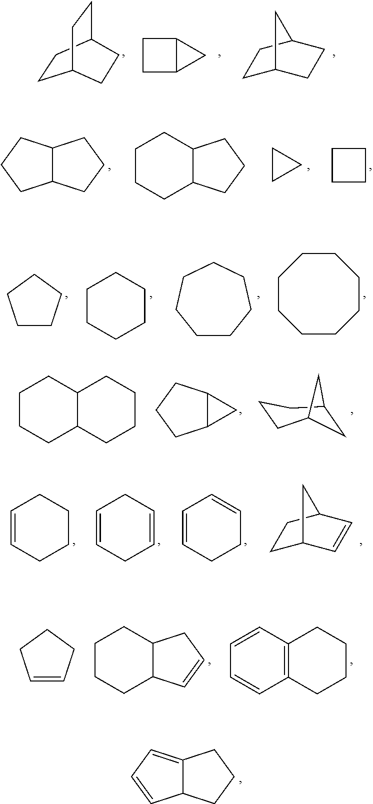

and the like.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “halo” or, alternatively, “halogen” means fluoro, chloro, bromo or iodo. Preferred halo groups are fluoro, chloro and bromo.

The terms “haloalkyl,” “haloalkenyl,” “haloalkynyl” and “haloalkoxy” include alkyl, alkenyl, alkynyl and alkoxy structures that are substituted with one or more halo groups or with combinations thereof. The terms “fluoroalkyl” and “fluoroalkoxy” include haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof.

The terms “heteroaryl” or, alternatively, “heteroaromatic” refers to an aryl group that includes one or more ring heteroatoms selected from nitrogen, oxygen and sulfur. An N-containing “hetero aromatic” or “heteroaryl” moiety refers to an aromatic group in which at least one of the skeletal atoms of the ring is a nitrogen atom. The polycyclic heteroaryl group may be fused or non-fused. Illustrative examples of heteroaryl groups include the following moieties:

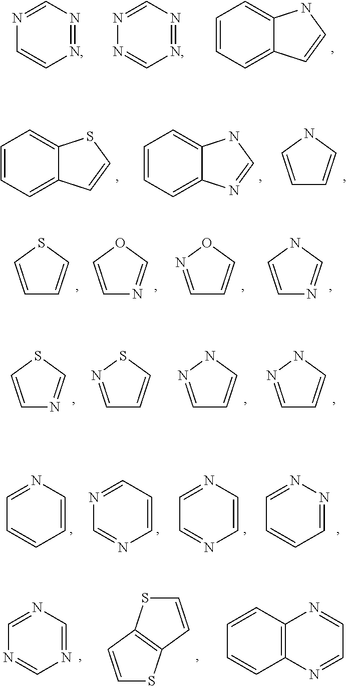

and the like.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

A “heteroalicyclic” group refers to a cycloalkyl group that includes at least one heteroatom selected from nitrogen, oxygen and sulfur. The radicals may be fused with an aryl or heteroaryl. Illustrative examples of heterocycloalkyl groups include:

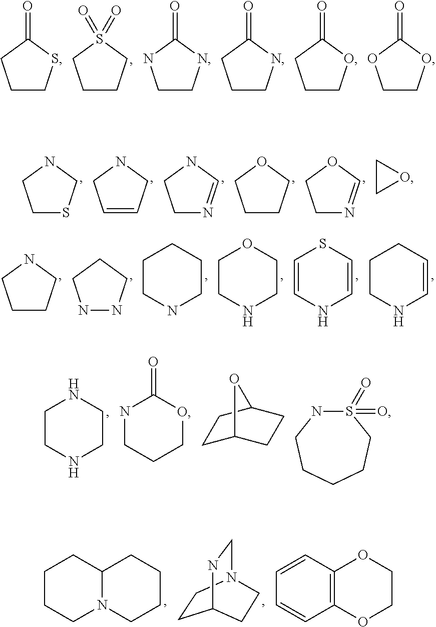

and the like. The term heteroalicyclic also includes all ring forms of the carbohydrates, including but not limited to the monosaccharides, the disaccharides and the oligosaccharides.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

An “isocyanato” group refers to a —NCO group.

An “isothiocyanato” group refers to a —NCS group.

A “mercaptyl” group refers to a (alkyl)S— group.

The terms “nucleophile” and “electrophile” as used herein have their usual meanings familiar to synthetic and/or physical organic chemistry. Carbon electrophiles typically comprise one or more alkyl, alkenyl, alkynyl or aromatic (sp³, sp², or sp hybridized) carbon atoms substituted with any atom or group having a Pauling electronegativity greater than that of carbon itself. Examples of carbon electrophiles include but are not limited to carbonyls (aldehydes, ketones, esters, amides), oximes, hydrazones, epoxides, aziridines, alkyl-, alkenyl-, and aryl halides, acyls, sulfonates (aryl, alkyl and the like). Other examples of carbon electrophiles include unsaturated carbon atoms electronically conjugated with electron withdrawing groups, examples being the 6-carbon in alpha-unsaturated ketones or carbon atoms in fluorine substituted aryl groups. Methods of generating carbon electrophiles, especially in ways which yield precisely controlled products, are known to those skilled in the art of organic synthesis.

The relative disposition of aromatic substituents (ortho, meta, and para) imparts distinctive chemistry for such stereoisomers and is well recognized within the field of aromatic chemistry. Para- and meta-substitutional patterns project the two substituents into different orientations. Ortho-disposed substituents are oriented at 60° with respect to one another; meta-disposed substituents are oriented at 120° with respect to one another; para-disposed substituents are oriented at 180° with respect to one another.

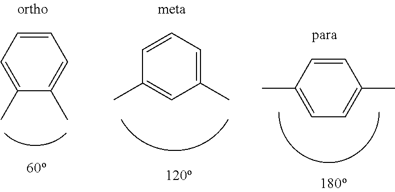

Relative dispositions of substituents, viz, ortho, meta, para, also affect the electronic properties of the substituents. Without being bound to any particular type or level of theory, it is known that ortho- and para-disposed substituents electronically affect one another to a greater degree than do corresponding meta-disposed substituents. Meta-disubstituted aromatics are often synthesized using different routes than are the corresponding ortho and para-disubstituted aromatics.

The term “moiety” refers to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

A “sulfinyl” group refers to a —S(═O)—R, where R is selected from the group consisting of alkyl, cycloalkyl, aryl, heteroaryl (bonded through a ring carbon) and heteroalicyclic (bonded through a ring carbon)

A “sulfonyl” group refers to a —S(═O)₂—R, where R is selected from the group consisting of alkyl, cycloalkyl, aryl, heteroaryl (bonded through a ring carbon) and heteroalicyclic (bonded through a ring carbon)

A “thiocyanato” group refers to a —CNS group.

The term “optionally substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. The protecting groups that may form the protective derivatives of the above substituents are known to those of skill in the art and may be found in references such as Greene and Wuts, above.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (I), as well as active metabolites of these compounds having the same type of activity. By way of example only, a known metabolite of fenretinide is N-(4-methoxyphenyl)retinamide, also known as 4-MPR or MPR. Another known metabolite of fenretinide is 4-oxo fenretinide. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

### Pharmaceutical Compositions

Another aspect are pharmaceutical compositions comprising a compound of Formula (I) and a pharmaceutically acceptable diluent, excipient, or carrier.

The term “pharmaceutical composition” refers to a mixture of a compound of Formula (I) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “carrier” refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The term “physiologically acceptable” refers to a material, such as a carrier or diluent, that does not abrogate the biological activity or properties of the compound, and is nontoxic.

The term “pharmaceutically acceptable salt” refers to a formulation of a compound that does not cause significant irritation to an organism to which it is administered and does not abrogate the biological activity and properties of the compound. Pharmaceutically acceptable salts may be obtained by reacting a compound of Formula (I) with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable salts may also be obtained by reacting a compound of Formula (I) with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods known in the art

A “metabolite” of a compound disclosed herein is a derivative of that compound that is formed when the compound is metabolized. The term “active metabolite” refers to a biologically active derivative of a compound that is formed when the compound is metabolized. The term “metabolized” refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

By way of example only, MPR is a known metabolite of HPR, both of which are contained within the structure of Formula (I). MPR accumulates systemically in patients that have been chronically treated with HPR. One of the reasons that MPR accumulates systemically is that MPR is only (if at all) slowly metabolized, whereas HPR is metabolized to MPR. In addition, MPR may undergo relatively slow clearance. Thus, (a) the pharmacokinetics and pharmacodynamics of MPR must be taken into consideration when administering and determining the bioavailability of HPR, (b) MPR is more stable to metabolism than HPR, and (c) MPR can be more immediately bioavailable than HPR following absorption. Another known metabolite of fenretinide is 4-oxo fenretinide.

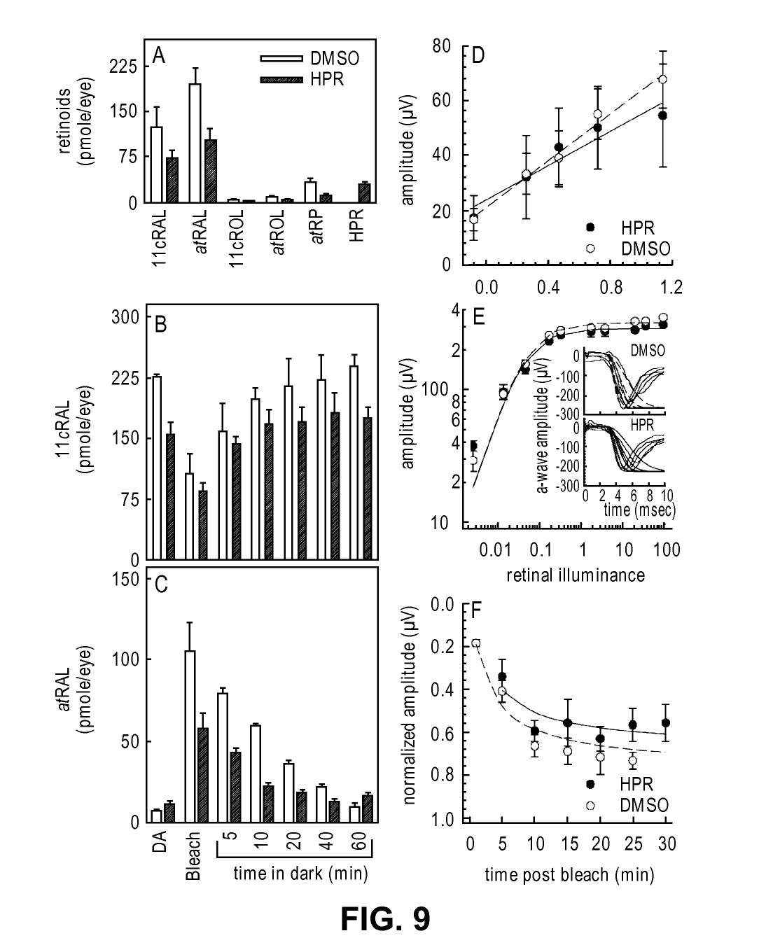

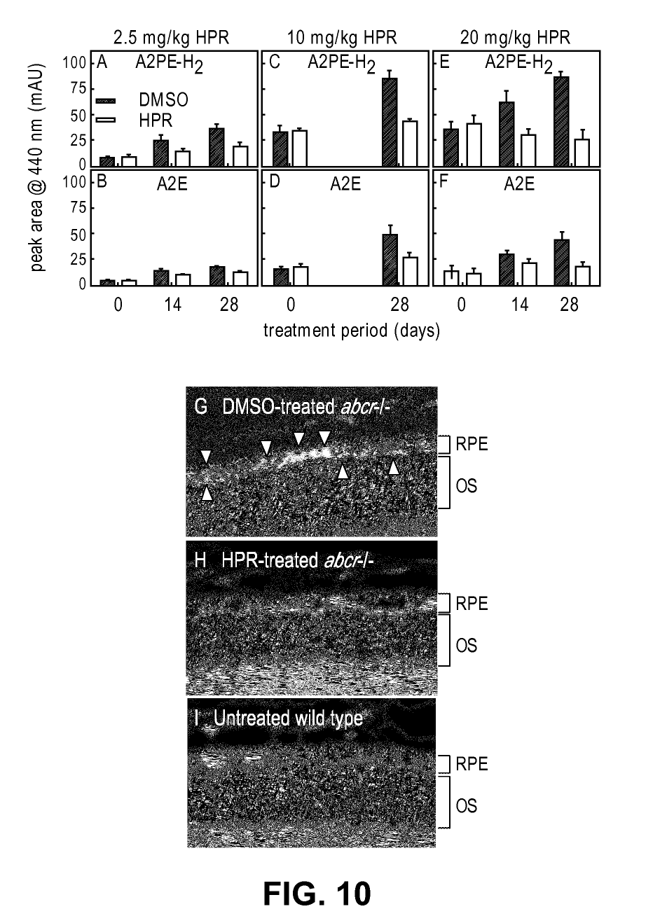

MPR may also be considered an active metabolite. As shown in FIGS. 9 and 10, MPR (like HPR) can bind to Retinol Binding Protein (RBP) and prevent the binding of RBP to Transerythrin (TTR). As a result, when either HPR or MPR is administered to a patient, one of the resulting expected features is that MPR will accumulate and bind to RBP and inhibit binding of retinol to RBP, as well as the binding of RBP to TTR. Accordingly, MPR can (a) serve as an inhibitor of retinol binding to RBP, (b) serve as an inhibitor of RBP to TTR, (c) limit the transport of retinol to certain tissues, including ophthalmic tissues, and (d) be transported by RBP to certain tissues, including ophthalmic tissues. MPR appears to bind more weakly to RBP than HPR, and is thus a less strong inhibitor of retinol binding to RBP. Nevertheless, both MPR and HPR are expected to inhibit, approximately equivalently, the binding of RBP to TTR. MPR has, in these respects, the same mode of action as HPR and can serve as a therapeutic agent in the methods and compositions described herein.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of Formula (I) which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The compounds described herein can be administered to a human patient per se, or in pharmaceutical compositions where they are mixed with other active ingredients, as in combination therapy, or suitable carrier(s) or excipient(s). Techniques for formulation and administration of the compounds of the instant application may be found in “Remington: The Science and Practice of Pharmacy,” 20th ed. (2000).

### Routes of Administration

Suitable routes of administration may, for example, include oral, rectal, transmucosal, transdermal, pulmonary, or intestinal administration; parenteral delivery, including intramuscular, subcutaneous, intravenous, intramedullary injections, as well as intrathecal, direct intraventricular, intraperitoneal, or intranasal injections.

Alternately, one may administer the compound in a local rather than systemic manner, for example, via injection of the compound directly into an organ, often in a depot or sustained release formulation. The liposomes will be targeted to and taken up selectively by the organ. In addition, the drug may be provided in the form of a rapid release formulation, in the form of an extended release formulation, or in the form of an intermediate release formulation.

### Composition/Formulation

Pharmaceutical compositions comprising a compound of Formula (I) may be manufactured in a manner that is itself known, e.g., by means of conventional mixing, dissolving, granulating, dragee-making, levigating, emulsifying, encapsulating, entrapping or compression processes.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

The compounds of Formula (I) can be administered in a variety of ways, including systemically, such as orally or intravenously.

A composition comprising a compound of Formula (I) can illustratively take the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition may include a gel formulation. In other embodiments, the liquid composition is aqueous. Alternatively, the composition can take the form of an ointment.

Useful aqueous suspension can also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Useful compositions can also comprise an acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful compositions may also include solubilizing agents to aid in the solubility of a compound of Formula (I). The term “solubilizing agent” generally includes agents that result in formation of a micellar solution or a true solution of the agent. Certain acceptable nonionic surfactants, for example polysorbate 80, can be useful as solubilizing agents, as can acceptable glycols, polyglycols, e.g., polyethylene glycol 400, and glycol ethers.

Useful compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Useful compositions may also include one or more acceptable salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful compositions may also include one or more acceptable preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions may include one or more acceptable surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions may include one or more antioxidants to enhance chemical stability where required. Suitable antioxidants include, by way of example only, ascorbic acid and sodium metabisulfite.

Aqueous suspension compositions can be packaged in single-dose non-reclosable containers. Alternatively, multiple-dose reclosable containers can be used, in which case it is typical to include a preservative in the composition.

For intravenous injections, compounds of Formula (I) may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

One useful formulation for solubilizing higher quantities of the compounds of Formula (I) are, by way of example only, positively, negatively or neutrally charged phospholipids, or bile salt/phosphatidylcholine mixed lipid aggregate systems, such as those described in Li, C. Y., et al., Pharm. Res. 13:907-913 (1996). An additional formulation that can be used for the same purpose with compounds having the structure of Formula (I) involves use of a solvent comprising an alcohol, such as ethanol, in combination with an alkoxylated caster oil. See, e.g., U.S. Patent Publication Number 2002/0183394. Or, alternatively, a formulation comprising a compound of Formula (I) is an emulsion composed of a lipoid dispersed in an aqueous phase, a stabilizing amount of a non-ionic surfactant, optionally a solvent, and optionally an isotonic agent. See id. Yet another formulation comprising a compound of Formula (I) includes corn oil and a non-ionic surfactant. See U.S. Pat. No. 4,665,098. Still another formulation comprising a compound of Formula (I) includes lysophosphatidylcholine, monoglyceride and a fatty acid. See U.S. Pat. No. 4,874,795. Still another formulation comprising a compound of Formula (I) includes flour, a sweetener, and a humectant. See International Publication No. WO 2004/069203. And still another formulation comprising a compound of Formula (I) includes dimyristoyl phosphatidylcholine, soybean oil, t-butyl alcohol and water. See U.S. Patent Application Publication No. US 2002/0143062.

For oral administration, compounds of Formula (I) can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated. Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, including by way of example only, soft, sealed capsules made of gelatin and a plasticizer, such as glycerol or sorbitol; or hard-gel capsules or tablets. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner.

Another useful formulation for administration of compounds having the structure of Formula (I) employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252. Such patches may be constructed for continuous, pulsatile, or on demand delivery of pharmaceutical agents. Still further, transdermal delivery of the compounds of Formula (I) can be accomplished by means of iontophoretic patches and the like. Transdermal patches can provide controlled delivery of the compounds. The rate of absorption can be slowed by using rate-controlling membranes or by trapping the compound within a polymer matrix or gel. Conversely, absorption enhancers can be used to increase absorption. Formulations suitable for transdermal administration can be presented as discrete patches and can be lipophilic emulsions or buffered, aqueous solutions, dissolved and/or dispersed in a polymer or an adhesive.

For administration by inhalation, the compounds of Formula (I) are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as rectal gels, rectal foam, rectal aerosols, suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Injectable depot forms may be made by forming microencapsulated matrices (also known as microencapsule matrices) of the compound of Formula (I) in biodegradable polymers. Depending upon the ratio of drug to polymer and the nature of the particular polymer employed, the rate of drug release can be controlled. Depot injectable formulations may be also prepared by entrapping the drug in liposomes or microemulsions. By way of example only, posterior juxtascleral depots may be used as a mode of administration for compounds having the structure of Formula (I). The sclera is a thin avascular layer, comprised of highly ordered collagen network surrounding most of vertebrate eye. Since the sclera is avascular it can be utilized as a natural storage depot from which injected material cannot rapidly removed or cleared from the eye. The formulation used for administration of the compound into the scleral layer of the eye can be any form suitable for application into the sclera by injection through a cannula with small diameter suitable for injection into the scleral layer. Examples for injectable application forms are solutions, suspensions or colloidal suspensions.

A pharmaceutical carrier for the hydrophobic compounds of Formula (I) is a cosolvent system comprising benzyl alcohol, a nonpolar surfactant, a water-miscible organic polymer, and an aqueous phase. The cosolvent system may be a 10% ethanol, 10% polyethylene glycol 300, 10% polyethylene glycol 40 castor oil (PEG-40 castor oil) with 70% aqueous solution. This cosolvent system dissolves hydrophobic compounds well, and itself produces low toxicity upon systemic administration. Naturally, the proportions of a cosolvent system may be varied considerably without destroying its solubility and toxicity characteristics. Furthermore, the identity of the cosolvent components may be varied: for example, other low-toxicity nonpolar surfactants may be used instead of PEG-40 castor oil, the fraction size of polyethylene glycol 300 may be varied; other biocompatible polymers may replace polyethylene glycol, e.g., polyvinyl pyrrolidone; and other sugars or polysaccharides maybe included in the aqueous solution.

Alternatively, other delivery systems for hydrophobic pharmaceutical compounds may be employed. Liposomes and emulsions are well known examples of delivery vehicles or carriers for hydrophobic drugs. Certain organic solvents such as N-methylpyrrolidone also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

One formulation for the administration of compounds having the structure of Formula (I) has been used with fenretinide in the treatment of neuroblastoma, prostate and ovarian cancers, and is marketed by Avanti Polar Lipids, Inc. (Alabaster, Ala.) under the name Lym-X-Sorb™. This formulation, which comprises an organized lipid matrix that includes lysophosphatidylcholine, monoglyceride and fatty acid, is designed to improve the oral availability of fenretinide. Such a formulation, i.e., an oral formulation that includes lysophosphatidylcholine, monoglyceride and fatty acid, is proposed to also provide improved bioavailability of compounds having the structure of Formula (I) for the treatment of ophthalmic and ocular diseases and conditions, including but not limited to the macular degenerations and dystrophies. This formulation can be used in a range of orally-administered compositions, including by way of example only, a capsule and a powder that can be suspended in water to form a drinkable composition.

All of the formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

Many of the compounds of Formula (I) may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

### Treatment Methods, Dosages and Combination Therapies

The term “mammal” means all mammals including humans. Mammals include, by way of example only, humans, non-human primates, cows, dogs, cats, goats, sheep, pigs, rats, mice and rabbits.

The term “effective amount” as used herein refers to that amount of the compound being administered which will relieve to some extent one or more of the symptoms of the disease, condition or disorder being treated.

The compositions containing the compound(s) described herein can be administered for prophylactic and/or therapeutic treatments. The term “treating” is used to refer to either prophylactic and/or therapeutic treatments. In therapeutic applications, the compositions are administered to a patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. It is considered well within the skill of the art for one to determine such therapeutically effective amounts by routine experimentation (e.g., a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial).

The terms “enhance” or “enhancing” means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system. When used in a patient, amounts effective for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease condition and its severity, the identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day, preferably 1-1500 mg per day. The desired dose may conveniently be presented in a single dose or as divided doses administered simultaneously (or over a short period of time) or at appropriate intervals, for example as two, three, four or more sub-doses per day.

In certain instances, it may be appropriate to administer at least one of the compounds described herein (or a pharmaceutically acceptable salt, ester, amide, prodrug, or solvate) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for macular degeneration involving administration of one of the compounds described herein, increased therapeutic benefit may result by also providing the patient with other therapeutic agents or therapies for macular degeneration. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of at least one compound of formula (I) with nitric oxide (NO) inducers, statins, negatively charged phospholipids, anti-oxidants, minerals, anti-inflammatory agents, anti-angiogenic agents, matrix metalloproteinase inhibitors, and carotenoids. In several instances, suitable combination agents may fall within multiple categories (by way of example only, lutein is an anti-oxidant and a carotenoid). Further, the compounds of Formula (I) may also be administered with additional agents that may provide benefit to the patient, including by way of example only cyclosporin A.

In addition, the compounds of Formula (I) may also be used in combination with procedures that may provide additional or synergistic benefit to the patient, including, by way of example only, the use of extracorporeal rheopheresis (also known as membrane differential filtration), the use of implantable miniature telescopes, laser photocoagulation of drusen, and microstimulation therapy.

The use of anti-oxidants has been shown to benefit patients with macular degenerations and dystrophies. See, e.g., Arch. Ophthalmol., 119: 1417-36 (2001); Sparrow, et al., J. Biol. Chem., 278:18207-13 (2003). Examples of suitable anti-oxidants that could be used in combination with at least one compound having the structure of Formula (I) include vitamin C, vitamin E, beta-carotene and other carotenoids, coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

The use of certain minerals has also been shown to benefit patients with macular degenerations and dystrophies. See, e.g., Arch. Ophthalmol., 119: 1417-36 (2001). Examples of suitable minerals that could be used in combination with at least one compound having the structure of Formula (I) include copper-containing minerals, such as cupric oxide (by way of example only); zinc-containing minerals, such as zinc oxide (by way of example only); and selenium-containing compounds.

The use of certain negatively-charged phospholipids has also been shown to benefit patients with macular degenerations and dystrophies. See, e.g., Shaban & Richter, Biol. Chem., 383:537-45 (2002); Shaban, et al., Exp. Eye Res., 75:99-108 (2002). Examples of suitable negatively charged phospholipids that could be used in combination with at least one compound having the structure of Formula (I) include cardiolipin and phosphatidylglycerol. Positively-charged and/or neutral phospholipids may also provide benefit for patients with macular degenerations and dystrophies when used in combination with compounds having the structure of Formula (I).

The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a very high number of apoptotic photoreceptor cells, while the group with high zeaxanthin concentrations had minimal damage. Examples of suitable carotenoids for in combination with at least one compound having the structure of Formula (I) include lutein and zeaxanthin, as well as any of the aforementioned carotenoids.

Suitable nitric oxide inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the endothelium, and has been identified as nitric oxide or a closely related derivative thereof (Palmer et al, Nature, 327:524-526 (1987); Ignarro et al, Proc. Natl. Acad. Sci. USA, 84:9265-9269 (1987)).

Statins serve as lipid-lowering agents and/or suitable nitric oxide inducers. In addition, a relationship has been demonstrated between statin use and delayed onset or development of macular degeneration. G. McGwin, et al., British Journal of Ophthalmology, 87:1121-25 (2003). Statins can thus provide benefit to a patient suffering from an ophthalmic condition (such as the macular degenerations and dystrophies, and the retinal dystrophies) when administered in combination with compounds of Formula (I). Suitable statins include, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

Suitable anti-inflammatory agents with which the Compounds of Formula (I) may be used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, or Celebrex™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

Suitable matrix metalloproteinases (MMPs) inhibitors may also be administered in combination with compounds of Formula (I) in order to treat ophthalmic conditions or symptoms associated with macular or retinal degenerations. MMPs are known to hydrolyze most components of the extracellular matrix. These proteinases play a central role in many biological processes such as normal tissue remodeling, embryogenesis, wound healing and angiogenesis. However, excessive expression of MMP has been observed in many disease states, including macular degeneration. Many MMPs have been identified, most of which are multidomain zinc endopeptidases. A number of metalloproteinase inhibitors are known (see for example the review of MMP inhibitors by Whittaker M. et al, Chemical Reviews 99(9):2735-2776 (1999)). Representative examples of MMP Inhibitors include Tissue Inhibitors of Metalloproteinases (TIMPs) (e.g., TIMP-1, TIMP-2, TIMP-3, or TIMP-4), α₂-macroglobulin, tetracyclines (e.g., tetracycline, minocycline, and doxycycline), hydroxamates (e.g., BATIMASTAT, MARIMISTAT and TROCADE), chelators (e.g., EDTA, cysteine, acetylcysteine, D-penicillamine, and gold salts), synthetic MMP fragments, succinyl mercaptopurines, phosphonamidates, and hydroxaminic acids. Examples of MMP inhibitors that may be used in combination with compounds of Formula (I) include, by way of example only, any of the aforementioned inhibitors.

The use of antiangiogenic or anti-VEGF drugs has also been shown to provide benefit for patients with macular degenerations and dystrophies. Examples of suitable antiangiogenic or anti-VEGF drugs that could be used in combination with at least one compound having the structure of Formula (I) include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone-ru486), subtenon triamcinolone acetonide, intravitreal crystalline triamcinolone acetonide, Prinomastat (AG3340—synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis). Resveratrol, which can be extracted from walnuts or the skins of red grapes, has demonstrated anti-angiogenic activity and can be used as the second or additional agent for the combination therapies described herein. Furthermore, other trans-stilbene compounds are expected to exhibit similar activity.

Other pharmaceutical therapies that have been used to relieve visual impairment can be used in combination with at least one compound of Formula (I). Such treatments include but are not limited to agents such as Visudyne™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), ranibizumab (Genentech), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group plc), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporin A. See U.S. Patent Application Publication No. 20040092435.

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; we envision the use of multiple therapeutic combinations. By way of example only, a compound having the structure of Formula (I) may be provided with at least one antioxidant and at least one negatively charged phospholipid; or a compound having the structure of Formula (I) may be provided with at least one antioxidant and at least one inducer of nitric oxide production; or a compound having the structure of Formula (I) may be provided with at least one inducer of nitric oxide productions and at least one negatively charged phospholipid; and so forth.

In addition, the compounds of Formula (I) may also be used in combination with procedures that may provide additional or synergistic benefit to the patient. Procedures known, proposed or considered to relieve visual impairment include but are not limited to ‘limited retinal translocation’, photodynamic therapy (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

Further combinations that may be used to benefit an individual include using genetic testing to determine whether that individual is a carrier of a mutant gene that is known to be correlated with certain ophthalmic conditions. By way of example only, defects in the human ABCA4 gene are thought to be associated with five distinct retinal phenotypes including Stargardt disease, cone-rod dystrophy, age-related macular degeneration and retinitis pigmentosa. See e.g., Allikmets et al., Science, 277:1805-07 (1997); Lewis et al., Am. J. Hum. Genet., 64:422-34 (1999); Stone et al., Nature Genetics, 20:328-29 (1998); Allikmets, Am. J. Hum. Gen., 67:793-799 (2000); Klevering, et al, Ophthalmology, 111:546-553 (2004). In addition, an autosomal dominant form of Stargardt Disease is caused by mutations in the ELOV4 gene. See Karan, et al., Proc. Natl. Acad. Sci. (2005). Patients possessing any of these mutations are expected to find therapeutic and/or prophylactic benefit in the methods described herein.

In addition, compounds of Formula (I) or other agents that result in the reduction of serum retinol levels can be administered with (meaning before, during or after) agents that treat or alleviate side effects arising from serum retinol reduction. Such side effects include dry skin and dry eye. Accordingly, agents that alleviate or treat either dry skin or dry eye may be administered with compounds of Formula (I) or other agents that reduce serum retinol levels.

Modulation of Vitamin A levels

Vitamin A (all-trans retinol) is a vital cellular nutrient which cannot be synthesized de novo and therefore must be obtained from dietary sources. Vitamin A is a generic term which may designate any compound possessing the biological activity, including binding activity, of retinol. One retinol equivalent (RE) is the specific biologic activity of 1 μg of all-trans retinol (3.33 IU) or 6 μg (10 IU) of beta-carotene. Beta-carotene, retinol and retinal (vitamin A aldehyde) all possess effective and reliable vitamin A activity. Each of these compounds are derived from the plant precursor molecule, carotene (a member of a family of molecules known as carotenoids). Beta-carotene, which consists of two molecules of retinal linked at their aldehyde ends, is also referred to as the provitamin form of vitamin A.

Ingested β-carotene is cleaved in the lumen of the intestine by β-carotene dioxygenase to yield retinal. Retinal is reduced to retinol by retinaldehyde reductase, an NADPH requiring enzyme within the intestines, and thereafter esterified to palmitic acid.

Following digestion, retinol in food material is transported to the liver bound to lipid aggregates. See Bellovino et al., Mol. Aspects. Med., 24:411-20 (2003). Once in the liver, retinol forms a complex with retinol binding protein (RBP) and is then secreted into the blood circulation. Before the retinol-RBP holoprotein can be delivered to extra-hepatic target tissues, such as by way of example, the eye, it must bind with transthyretin (TTR). Zanotti and Berni, Vitam. Horm., 69:271-95 (2004). It is this secondary complex which allows retinol to remain in the circulation for prolonged periods. Association with TTR facilitates RBP release from hepatocytes, and prevents renal filtration of the RBP-retinol complex. The retinol-RBP-TTR complex is delivered to target tissues where retinol is taken up and utilized for various cellular processes. Delivery of retinol to cells through the circulation by the RBP-TTR complex is the major pathway through which cells and tissue acquire retinol.

Retinol uptake from its complexed retinol-RBP-TTR form into cells occurs by binding of RBP to cellular receptors on target cells. This interaction leads to endocytosis of the RBP-receptor complex and subsequent release of retinol from the complex, or binding of retinol to cellular retinol binding proteins (CRBP), and subsequent release of apoRBP by the cells into the plasma. Other pathways contemplate alternative mechanisms for the entry of retinol into cells, including uptake of retinol alone into the cell. See Blomhoff (1994) for review.

The methods and compositions described herein are useful for the modulation of vitamin A levels in a mammalian subject. In particular, modulation of vitamin A levels can occur through the regulation of retinol binding protein (RBP) and transthyretin (TTR) availability in a mammal. The methods and compositions described herein provide for the modulation of RBP and TTR levels in a mammalian subject, and subsequently modulation of vitamin A levels. Increases or decreases in vitamin A levels in a subject can have effects on retinol availability in target organs and tissues. Therefore, providing a means of modulating retinol or retinol derivative availability may correspondingly modulate disease conditions caused by a lack of or excess in local retinol or retinol derivative concentrations in the target organs and tissues.

For example, A2E, the major fluorophore of lipofuscin, is formed in macular or retinal degeneration or dystrophy, including age-related macular degeneration and Stargardt Disease, due to excess production of the visual-cycle retinoid, all-trans-retinaldehyde, a precursor of A2E. Reduction of vitamin A and all-trans retinaldehyde in the retina, therefore, would be beneficial in reducing A2E and lipofuscin build-up, and treatment of age-related macular degeneration. Studies have confirmed that reducing serum retinol may have a beneficial effect of reducing A2E and lipofuscin in RPE. For example, animals maintained on a vitamin A deficient diet have been shown to demonstrate significant reductions in lipofuscin accumulation. Katz et al., Mech. Ageing Dev., 35:291-305 (1986); Katz et al., Mech. Ageing Dev., 39:81-90 (1987); Katz et al., Biochim. Biophys. Acta, 924:432-41 (1987). Further evidence that reducing vitamin A levels may be beneficial in the progression of macular degeneration and dystrophy was shown by Radu and colleagues, where reduction in ocular vitamin A levels resulted in reductions in both lipofuscin and A2E. Radu et al., Proc. Natl. Acad. Sci. USA, 100:4742-7 (2003); Radu et al., Proc. Natl. Acad. Sci. USA, 101:5928-33 (2004).

Administration of the retinoic acid analog, N-4-(hydroxyphenyl)retinamide (HPR or fenretinide), has been shown to cause reductions in serum retinol and RBP. Formelli et al., Cancer Res. 49:6149-52 (1989); Formelli et al., J. Clin Oncol., 11:2036-42 (1993); Torrisi et al., Cancer Epidemiol. Biomarkers Prev., 3:507-10 (1994). In vitro studies have demonstrated that HPR interferes with the normal interaction of TTR with RBP. Malpeli et al., Biochim. Biophys. Acta 1294: 48-54 (1996); Holven et al., Int. J. Cancer 71:654-9 (1997).

Modulators (e.g. HPR) that inhibit delivery of retinol to cells either through interruption of binding of retinol to apo RBP or holo RBP (RBP+retinol) to its transport protein, TTR, or the increased renal excretion of RBP and TTR, therefore, would be useful in decreasing serum vitamin A levels, and buildup of retinol and its derivatives in target tissues such as the eye.

Similarly, modulators which reduce the availability of the retinol transport proteins, retinol binding protein (RBP) and transthyretin (TTR), would also be useful in decreasing serum vitamin A levels, and buildup of retinol and its derivatives and physical manifestations in target tissues, such as the eye. TTR, for example, has been shown to be a component of Drusen constituents, suggesting a direct involvement of TTR in age-related macular degeneration. Mullins, R F, FASEB J. 14:835-846 (2000); Pfeffer B A, et al., Molecular Vision 10:23-30 (2004).

One embodiment of the methods and compositions disclosed herein, therefore, provides for the modulation of RBP or TTR levels in a mammal by administering to a mammal at least once an effective amount of at least one of the compounds chosen from the group consisting of an RBP clearance agent, a TTR clearance agent, an RBP antagonist, an RBP agonist, a TTR antagonist, a TTR agonist and a retinol binding protein receptor antagonist.

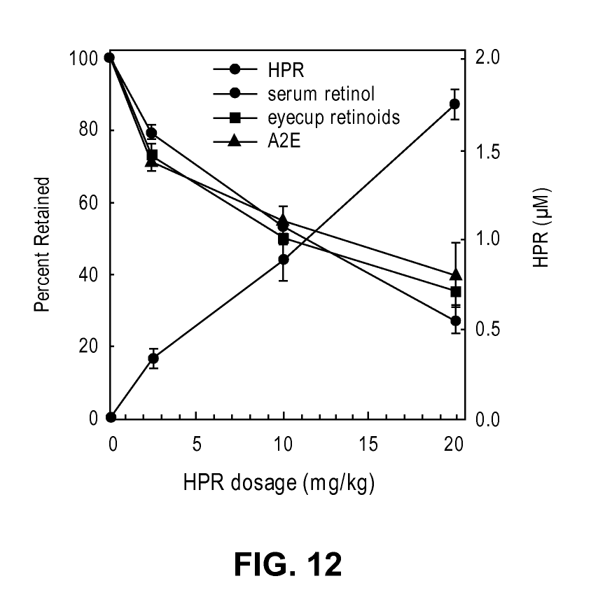

Regardless of the mechanism by which an agent reduces the level of serum retinol in a patient, such a reduction also provides a new approach to reducing the level of retinoids and the level of A2E in the eye of the mammal (see, e.g., Example 22 and FIG. 12). In essence, there is a clear and direct relationship between a reduction in the serum retinol and a reduction in the level of retinoids and the level of A2E in the eye of a mammal (FIG. 12). Serum retinol reduction can be used to treat any or all of the following: (a) ophthalmic diseases or conditions that arise from accumulation of A2E, N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, N-retinylidene-phosphatidylethanolamine, or retinoids in the eye; (b) juvenile macular degeneration, including Stargardt Disease; (c) a lipofuscin-based retinal disease; (d) dry form age-related macular degeneration; (e) cone-rod dystrophy; (f) retinitis pigmentosa; (g) wet-form age-related macular degeneration; (h) ophthalmic diseases or conditions that present geographic atrophy and/or photoreceptor degeneration; (i) ophthalmic diseases or conditions that present trans-retinal accumulation; (j) ophthalmic diseases or conditions that present a sensitivity to light; (k) ophthalmic diseases or conditions that present drusen formation; (l) ophthalmic diseases or conditions that result from the overactivity of a visual cycle protein (including transport/chaperone proteins); (m) ophthalmic diseases or conditions that present liposfuscin accumulation; or (n) lipofuscin-based retinal degeneration. Further, such treatments for ophthalmic diseases or conditions can be effected without directly inhibiting (i.e., binding to) a visual cycle protein, visual cycle ligand, or other component of the visual cycle. However, if desired, the use of a second agent that inhibits one of the visual cycle proteins may be useful for additional and/or synergistic effects in the treatment of ophthalmic diseases or conditions. Use of an agent that lowers and/or modulates serum retinol levels may have additional advantages, such as broadly depleting total ocular retinoid concentrations without necessitating high intraocular concentrations of inhibitors for specific proteins or transport proteins.

### Retinol Binding Protein (RBP) and Transthyretin (TTR)

Retinol binding protein, or RBP, is a single polypeptide chain, with a molecular weight of approximately 21 kD. RBP has been cloned and sequenced, and its amino acid sequence determined. Colantuni et al., Nuc. Acids Res., 11:7769-7776 (1983). The three-dimensional structure of RBP reveals a specialized hydrophobic pocket designed to bind and protect the fat-soluble vitamin retinol. Newcomer et al., EMBO J., 3:1451-1454 (1984). In in vitro experiments, cultured hepatocytes have been shown to synthesize and secrete RBP. Blaner, W. S., Endocrine Rev., 10:308-316 (1989). Subsequent experiments have demonstrated that many cells contain mRNA for RBP, suggesting a widespread distribution of RBP synthesis throughout the body. See Blaner (1989). Most of the RBP secreted by the liver contains retinol in a 1:1 molar ratio, and retinol binding to RBP is required for normal RBP secretion.

In cells, RBP tightly binds to retinol in the endoplasmic reticulum, where it is found in high concentrations. Binding of retinol to RBP initiates a translocation of retinol-RBP from endoplasmic reticulum to the Golgi complex, followed by secretion of retinol-RBP from the cells. RBP secreted from hepatocytes also assists in the transfer of retinol from hepatocytes to stellate cells, where direct secretion of retinol-RBP into plasma takes place.

In plasma, approximately 95% of the plasma RBP is associated with transthyretin (TTR) in a 1:1 mol/mol ratio, wherein essentially all of the plasma vitamin A is bound to RBP. TTR is a well-characterized plasma protein consisting of four identical subunits with a molecular weight of 54,980. The full three-dimensional structure, elucidated by X-ray diffraction, reveals extensive β-sheets arranged tetrahedrally. Blake et al., J. Mol. Biol., 121:339-356 (1978). A channel runs through the center of the tetramer in which is located two binding sites for thyroxine. However, only one thyroxine molecule appears to be bound normally to TTR due to negative cooperativity. The complexation of TTR to RBP-retinol is thought to reduce the glomerular filtration of retinol, thereby increasing the half-life of retinol and RBP in plasma by about threefold.

### Modulation of RBP or TTR Binding or Clearance in a Subject

Before retinol bound to RBP is transported in the blood stream for delivery to the eye, it must be complexed with TTR. It is this secondary complex which allows retinol to remain in the circulation for prolonged periods. In the absence of TTR, the retinol-RBP complex would be rapidly excreted in the urine. Similarly, in the absence of RBP, retinol transport in the blood stream and uptake by cells would be diminished.

Another embodiment of the invention, therefore, is to modulate availability of RBP or TTR for complexing to retinol or retinol-RBP in the blood stream by modulating RBP or TTR binding characteristics or clearance rates. As mentioned above, the TTR binding to RBP holoprotein decreases the clearance rate of RBP and retinol. Therefore, by modulating either RBP or TTR availability, retinol levels may likewise be modulated in a subject in need thereof.

For example, antagonists of retinol binding to RBP may be used in the methods and compositions disclosed herein. An antagonist of retinol binding to RBP may include retinol derivatives or analogs which compete with the binding of retinol to RBP. Alternatively, an antagonist may comprise a fragment of an RBP which competes with native RBP for retinol binding, but does not allow retinol delivery to cells. This may include regions important for RBP binding to retinol binding protein receptor on cells. Alternatively, or in addition to, an immunoglobulin capable of binding to RBP or another protein, for example, on the cell surface, may be used so long as it interferes with the ability of RBP to bind to retinol and/or the uptake of retinol by the binding of RBP to retinol binding protein receptor. As above, the immunoglobulin may be a monoclonal or a polyclonal antibody.

As mentioned above, one means by which RBP binding to retinol may be modulated is to competitively bind RBP agonists or antagonists, such as retinol analogues. Therefore, one embodiment of the methods and compositions disclosed herein provides for RBP agonists or RBP antagonists in modulating RBP levels. For example, administration of the retinoic acid analog, N-4-(hydroxyphenyl)retinamide (HPR or fenretinide), has been shown to cause profound reductions in serum retinol and RBP. Formelli et al., Cancer Res. 49:6149-52 (1989); Formelli et al., J. Clin Oncol., 11:2036-42 (1993); Torrisi et al., Cancer Epidemiol. Biomarkers Prev., 3:507-10 (1994). In vitro studies have demonstrated that HPR interferes with the normal interaction of TTR with RBP. See Malpeli et al., Biochim. Biophys. Acta 1294: 48-54 (1996); Holven et al., Int. J. Cancer 71:654-9 (1997).

Further potential modulators of RBP levels include, by way of example (additional embodiments are noted herein and new embodiments may be selected using the screening methods and assays described herein) compounds having the structure of Formula (I). Fenretinide (hereinafter referred to as hydroxyphenyl retinamide) is one example of a compound having the structure of Formula (I) and is particularly useful in the compositions and methods disclosed herein. As will be explained below, fenretinide may be used as a modulator of retinol-RBP binding. In some aspects of the methods and compositions described herein, derivatives of fenretinide may be used instead of, or in combination with, fenretinide. As used herein, a “fenretinide derivative” refers to a compound whose chemical structure comprises a 4-hydroxy moiety and a retinamide.

In some embodiments, derivatives of fenretinide that may be used include, but are not limited to, C-glycoside and arylamide analogues of N-(4-hydroxyphenyl) retinamide-O-glucuronide, including but not limited to 4-(retinamido)phenyl-C-glucuronide, 4-(retinamido)phenyl-C-glucoside, 4-(retinamido)phenyl-C-xyloside, 4-(retinamido)benzyl-C-glucuronide, 4-(retinamido)benzyl-C-glucoside, 4-(retinamido)benzyl-C-xyloside; and retinoyl β-glucuronide analogues such as, for example, 1-(β-D-glucopyranosyl) retinamide and 1-(D-glucopyranosyluronosyl) retinamide, described in U.S. Pat. Nos. 5,516,792, 5,663,377, 5,599,953, 5,574,177, and Bhatnagar et al., Biochem. Pharmacol., 41:1471-7 (1991), each incorporated herein by reference.

Similarly, modulation of TTR binding may occur with competitive binders to TTR ligand binding, such as thyroxine or tri-iodothyronine or their respective analogs, or to RBP binding on TTR. TTR is a tetrameric protein comprised of identical 127 amino acid β-sheet sandwich subunits, and its three-dimensional configuration is known. Blake, C., et al., J. Mol. Biol. 61:217-224 (1971); Blake, C. et al., J. Mol. Biol. 121:339-356 (1978). TTR complexes to holo-RBP, and increase retinol and RBP half-lives by preventing glomerular filtration of RBP and retinol. Modulating TTR binding to holo RBP, therefore, may modulate RBP and retinol levels by decreasing the half-life of these compositions.

The three-dimensional structure of TTR complexed with holo RBP shows that TTR's natural ligand, thyroxine, does not interfere with binding to RBP holoprotein. Monaco, H. L., et al. Science, 268:1039-1041 (1995). However, studies involving competitive inhibitors to thyroxine binding have shown that disruption of the TTR-RBP holoprotein complex can occur, resulting in decrease plasma retinol levels in the subject. For example, metabolites to 3,4,3′,4′-tetrachlorobiphenyl reduces RBP binding sites on TTR, and inhibits formation of the TTR-RBP holoprotein complex. See Brouwer, A., et al. Chem. Biol. Interact., 68:203-17 (1988); Brouwer, A., et al., Toxicol. Appl. Pharmacol. 85:310-312 (1986). Therefore, one embodiment of the methods and compositions disclosed herein include the use of hydroxylated polyhalogenated aromatic hydrocarbon metabolites for the modulation of TTR or RBP availability.

By way of example only, other TTR modulators include diclofenac, a diclofenac analogue, a small molecule compound, an endocrine hormone analogue, a flavonoid, a non-steroidal anti-inflammatory drug, a bivalent inhibitor, a cardiac agent, a peptidomimetic, an aptamer, and an antibody.

In one embodiment, non-steroidal inflammatory agents may be used as TTR modulators, including but not limited to flufenamic acid, mefenamic acid, meclofenamic acid, diflunisal, diclofenac, diclofenamic acid, sulindac and indomethacin. See Peterson, S. A., et al., Proc. Natl. Acad. Sci. 95:12956-12960 (1998); Purkey, H. E., et al., Proc. Natl. Acad. Sci. 98:5566-5571 (2001), both of which are incorporated herein by reference in their entirety.

Diclofenac analogues may also be used in conjunction with the methods and compositions disclosed herein. Some examples include 2-[(2,6-dichlorophenyl)amino]benzoic acid; 2-[(3,5-dichlorophenyl)amino]benzoic acid; 3,5,-dichloro-4-[(4-nitrophenyl)amino]benzoic acid; 2-[(3,5-dichlorophenyl)amino]benzene acetic acid and 2-[(2,6-dichloro-4-carboxylic acid-phenyl)amino]benzene acetic acid. See Oza, V. B. et al., J. Med. Chem. 45:321-332 (2002), hereby incorporated by reference in its entirety. Similarly, diflunisal analogues may also be used in conjunction with the methods and compositions disclosed herein. Some examples include 3′,5′-difluorobiphenyl-3-ol; 2′,4′-difluorobiphenyl-3-carboxylic acid; 2′,4′-difluorobiphenyl-4-carboxylic acid; 2′-fluorobiphenyl-3-carboxylic acid; 2′-fluorobiphenyl-4-carboxylic acid; 3′,5′-difluorobiphenyl-3-carboxylic acid; 3′,5′-difluorobiphenyl-4-carboxylic acid; 2′,6′-difluorobiphenyl-3-carboxylic acid; 2′6′-difluorobiphenyl-4-carboxylic acid; biphenyl-4-carboxylic acid; 4′fluoro-4-hydroxybiphenyl-3-carboxylic acid; 2′-fluoro-4-hydroxybiphenyl-3-carboxylic acid; 3′,5′-difluoro-4-hydroxybiphenyl-3-carboxylic acid; 2′,4′-dichloro-4-hydroxybiphenyl-3-carboxylic acid; 4-hydroxybiphenyl-3-carboxylic acid; 3′5′-difluoro-4′hydroxybiphenyl-3-carboxylic acid; 3′,5′-difluoro-4′hydroxybiphenyl-4-carboxylic acid; 3′,5′-dichloro-4′hydroxybiphenyl-3-carboxylic acid; 3′,5′-dichloro-4′hydroxybiphenyl-4-carboxylic acid; 3′,5′-dichloro-3-formylbiphenyl; 3′,5′-dichloro-2-formylbiphenyl; 2′,4′-dichlorobiphenyl-3-carboxylic acid; 2′,4′-dichlorobiphenyl-4-carboxylic acid; 3′,5′-dichlorobiphenyl-3-yl-methanol; 3′,5′-dichlorobiphenyl-4-yl-methanol; or 3′,5′-dichlorobiphenyl-2-yl-methanol. See Adamski-Werner, S. L., et al., J. Med. Chem. 47:355-374 (2004), the teachings of which are hereby incorporated by reference in its entirety. Bivalent inhibitors, which link small molecule analogues into one compound, may also be used in conjunction with the methods and compositions disclosed herein. Green, N. S., et al., J. Am. Chem. Soc., 125:13404-13414 (2003).

Flavonoids and related compounds have also been shown to compete with thyroxine for binding to TTR. By way of example only, some flavonoids that may be used in conjunction with the methods and compositions disclosed herein include 3-methyl-4′,6-dihydroxy-3′,5′-dibromoflavone or 3′,5′-dibromo-2′,4,4′,6-tetrahydroxyaurone. Flavenoids and flavanoids, which are related to flavonoids, may also be used as modulators of TTR binding. In addition, cardiac agents have been shown to compete with thyroxine for binding to TTR. See Pedraza, P., et al., Endocrinology 137:4902-4914 (1996), herein incorporated by reference. These agents include, by way of example only, milrinone and aminone. See Davis, P J, et al., Biochem. Pharmacol. 36:3635-3640 (1987); Cody, V., Clin. Chem. Lab. Med. 40:1237-1243 (2002).

Additionally, hormone analogues, agonists and antagonists have been shown to be effective competitive inhibitors for thyroid hormone, including thyroxine and tri-iodothyronine. For example, diethylstilbestrol, an estrogen antagonist, has been shown to bind to and inhibit thyroxine binding. See Morais-de-Sa, E., et al., J. Biol. Chem. Epub. (Oct. 6, 2004), incorporated herein by reference in its entirety. Thyroxine-proprionic acid, thyroxine acetic acid and SKF-94901 are some examples of thyroxine analogs which may act as modulators of TTR binding. See Cody, V. (2002). In addition, retinoic acid has also been shown to inhibit thyroxine binding to human transthyretin. Smith, T J, et al., Biochim. Biophys. Acta, 1199:76 (1994).

Other embodiments include the use of small molecule inhibitors as modulators of TTR binding. Some examples include N-phenylanthranilic acid, methyl red, mordant orange I, bisarylamine, N-benzyl-p-aminobenzoic acid, furosamide, apigenin, resveratrol, dibenzofuran, niflumic acid, or sulindac. See Baures, P. W., et al. Bioorg. & Med. Chem. 6:1389-1401 (1998), incorporated by reference herein.

Modulators for use herein are also intended to include, a protein, polypeptide or peptide including, but not limited to, a structural protein, an enzyme, a cytokine (such as an interferon and/or an interleukin), an antibiotic, a polyclonal or monoclonal antibody, or an effective part thereof, such as an Fv fragment, which antibody or part thereof may be natural, synthetic or humanised, a peptide hormone, a receptor, a signalling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which may be modified or unmodified; an amino acid or analogue thereof, which may be modified or unmodified; a non-peptide (e.g., steroid) hormone; a proteoglycan; a lipid; or a carbohydrate. Small molecules, including inorganic and organic chemicals, which bind to and occupy the active site of the polypeptide thereby making the catalytic site inaccessible to substrate such that normal biological activity is prevented, are also included. Examples of small molecules include but are not limited to small peptides or peptide-like molecules.

### Detection of Modulator Activity

The compounds and compositions disclosed herein can also be used in assays for detecting perturbations in RBP or TTR availability through conventional means. For example, a subject may be treated with any of the compounds or compositions disclosed herein, and RBP or TTR levels quantified using conventional assay techniques. See Sundaram, M., et al., Biochem. J. 362:265-271 (2002). For example, a typical non-competitive sandwich assay is an assay disclosed in U.S. Pat. No. 4,486,530, incorporated herein by reference. In this method, a sandwich complex, for example an immune complex, is formed in an assay medium. The complex comprises the analyte, a first antibody, or binding member, that binds to the analyte and a second antibody, or binding member that binds to the analyte or a complex of the analyte and the first antibody, or binding member. Subsequently, the sandwich complex is detected and is related to the presence and/or amount of analyte in the sample. The sandwich complex is detected by virtue of the presence in the complex of a label wherein either or both the first antibody and the second antibody, or binding members, contain labels or substituents capable of combining with labels. The sample may be plasma, blood, feces, tissue, mucus, tears, saliva, or urine, for example for detecting modulation of clearance rates for RBP or TTR. For a more detailed discussion of this approach see U.S. Pat. Nos. Re 29,169 and 4,474,878, the relevant disclosures of which are incorporated herein by reference.

In a variation of the above sandwich assay, the sample in a suitable medium is contacted with labeled antibody or binding member for the analyte and incubated for a period of time. Then, the medium is contacted with a support to which is bound a second antibody, or binding member, for the analyte. After an incubation period, the support is separated from the medium and washed to remove unbound reagents. The support or the medium is examined for the presence of the label, which is related to the presence or amount of analyte. For a more detailed discussion of this approach see U.S. Pat. No. 4,098,876, the relevant disclosure of which is incorporated herein by reference.

The modulators disclosed herein may also be used in in vitro assays for detecting perturbations in RBP or TTR activity. For example, the modulator may be added to a sample comprising RBP, TTR and retinol to detect complex disruption. A component, for example, RBP, TTR, retinol or the modulator, may be labeled to determine if disruption of complex formation occurs. Complex formation and subsequent disruption may be detected and/or measured through conventional means, such as the sandwich assays disclosed above. Other detection systems may also be used to detect modulation of RBP or TTR binding, for example, FRET detection of RBP-TTR-retinol complex formation. See U.S. Provisional Patent Application No. 60/625,532 “Fluorescence Assay for Modulators of Retinol Binding,” herein incorporated by reference in its entirety.

In vitro gene expression assays may also be used to detect modulation of transcription or translation of RBP or TTR by the modulators disclosed herein. For example, as described in Wodicka et al., Nature Biotechnology 15 (1997), (hereby incorporated by reference in its entirety), because mRNA hybridization correlates to gene expression level, hybridization patterns can be compared to determine differential gene expression. As a non-limiting example, hybridization patterns from samples treated with the modulators may be compared to hybridization patterns from samples which have not been treated or which have been treated with a different compound or with different amounts of the same compound. The samples may be analyzed using DNA array technology, see U.S. Pat. No. 6,040,138, herein incorporated by reference in its entirety. Gene expression analysis of RBP or TTR activity may also be analyzed using recombinant DNA technology by analyzing the expression of reporter proteins driven by RBP or TTR promoter regions in an in vitro assay. See, e.g., Rapley and Walker, Molecular Biomethods Handbook (1998); Wilson and Walker, Principals and Techniques of Practical Biochemistry (2000), hereby incorporated by reference in its entirety.

In vitro translation assays may also be used to detect modulation or translation of RBP or TTR by the modulators disclosed herein. By way of example only, modulation of translation by the modulators may be detected through the use of cell-free protein translation systems, such as E. coli extract, rabbit reticulocyte lysate and wheat germ extract, see Spirin, A. S., Cell-free protein synthesis bioreactor (1991), herein incorporated by reference in its entirety, by comparing translation of proteins in the presence and absence of the modulators disclosed herein. Modulator effects on protein translation may also be monitored using protein gel electrophoretic or immune complex analysis to determine qualitative and quantitative differences after addition of the modulators.

In addition, other potential modulators which include, but are not limited to, small molecules, polypeptides, nucleic acids and antibodies, may also be screened using the in vitro detection methods described above. For example, the methods and compositions described herein may be used to screen small molecule libraries, nucleic acid libraries, peptide libraries or antibody libraries in conjunction with the teachings disclosed herein. Methods for screening libraries, such as combinatorial libraries and other libraries disclosed above, can be found in U.S. Pat. Nos. 5,591,646; 5,866,341; and 6,343,257, which are hereby incorporated by reference in its entirety.

### In Vivo Detection of Modulator Activity

In addition to the in vitro methods disclosed above, the methods and compositions disclosed herein may also be used in conjunction with in vivo detection and/or quantitation of modulator activity on TTR or RBP availability. For example, labeled TTR or RBP may be injected into a subject, wherein a candidate modulator added before, during or after the injection of the labeled TTR or RBP. The subject may be a mammal, for example a human; however other mammals, such as primates, horse, dog, sheep, goat, rabbit, mice or rats may also be used. A biological sample is then removed from the subject and the label detected to determine TTR or RBP availability. A biological sample may comprise, but is not limited to, plasma, blood, urine, feces, mucus, tissue, tears or saliva. Detection of the labeled reagents disclosed herein may take place using any of the conventional means known to those of ordinary skill in the art, depending upon the nature of the label. Examples of monitoring devices for chemiluminescence, radiolabels and other labeling compounds can be found in U.S. Pats. No. 4,618,485; 5,981,202, the relevant disclosures of which are herein incorporated by reference.

### HPR Mechanism of Action

HPR acts systemically to reduce retinoid content in the eye. HPR competes with dietary retinol for binding on RBP in the circulation. Once bound to RBP, HPR prevents complexation with TTR. TTR is a serum-borne protein which must complex with RBP-retinol in order to sustain high steady-state levels of RBP and retinol in the circulation. Consequently, the immediate effect of HPR treatment is reduced levels of RBP and retinol in serum. Unlike other extra-hepatic tissues which are able to uptake free retinol or retinyl esters from serum (e.g., kidney, testes, lung and adipose tissue), the RPE has a unique requirement for retinol delivered by RBP. Thus, the RPE is more susceptible to reductions in serum RBP-retinol than other tissues. The reduced transport of RBP-retinol to the RPE results in reduced retinoid flux through the visual cycle and, ultimately, reduced retinal fluorophores.

Effects of on HPR Visual Cycle Retinoids and Regeneration of Rhodopsin

While reducing serum RBP-retinol, HPR does not interact directly with enzymes and/or proteins of the visual cycle. This issue has been explored in a series of studies in which the effects of HPR on visual cycle retinoids were examined in vivo.

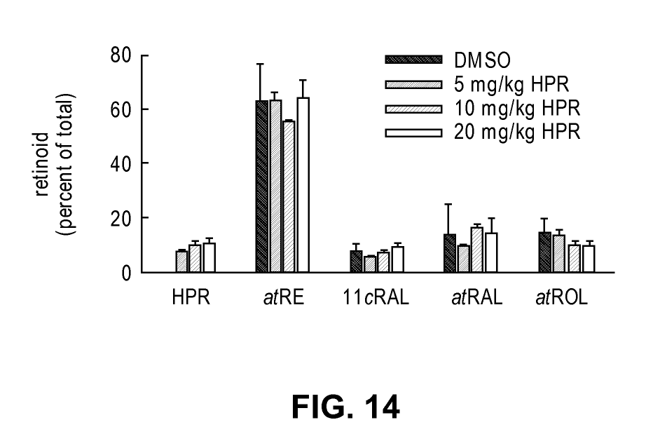

In one study, wild-type mice were given varied doses of HPR (5-20 mg/kg/day, i.p. in DMSO) for 7 days. Control mice received only DMSO. Mice were maintained on a 12 h/12 h light/dark cycle throughout the treatment period. At the end of the study, the ocular retinoid content was determined by high-performance liquid chromatography (HPLC). Light-adapted, rather than dark-adapted, retinoid profiles were obtained so that a measure of retinoids could be obtained while the visual cycle was actively regenerating chromophore. The data revealed a modest accumulation of HPR (4-6 μM) within the RPE in a dose-dependent manner. However, despite the presence of HPR within RPE cells, there were no significant differences in the light-adapted retinoid levels throughout the dosage regime (FIG. 14). These data indicate that HPR does not have a direct effect on retinoid biosynthesis within the visual cycle. This finding is in sharp contrast to data obtained from analysis of mice treated with 13-cis retinoic acid. In this study (Radu R A, et al., Proc Natl Acad Sci USA. 2003; 100(8): 4742-4747), the levels of 11-cis retinal were significantly reduced by increased doses of 13-cis retinoic acid. In addition, 11-cis retinyl esters, which are barely detectable in untreated mice, increased dramatically with increased 13-cis retinoic acid. These results are what would be predicted by inhibiting 11cRDH activity. Reduced 11cRDH activity would lead to reduced levels of 11-cis retinal and accumulation of 11-cis retinol. Free 11-cis retinol would be rapidly esterified via LRAT activity resulting in increased 11-cis retinyl esters. This effect of 13-cis retinoic acid on retinoid biosynthesis was also observed in rats in Sieving P A, et al., Proc Natl Acad Sci USA. 2001; 98(4): 1835-40.

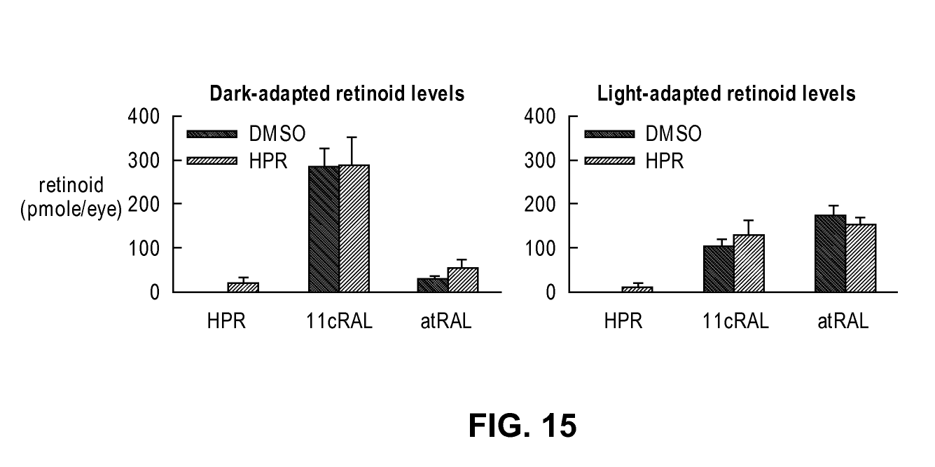

The effect of HPR on visual chromophore biosynthesis was examined in a separate study in which a single dose of HPR (10 mg/kg/day) was administered to abca-4−/− mice over a 7-day period. HPLC analysis of HPR and retinaldehyde content in both dark- and light-adapted mice confirmed that the presence of HPR within ocular tissues had essentially no effect on either steady-state retinal levels or regeneration of visual chromophore (FIG. 15).

Chronic HPR Administration Reduces Visual Cycle Retinoids but Does not Affect the Rate of Rhodopsin Regeneration

A number of biochemical and physiological studies demonstrate that short-term HPR treatment (7 days) caused essentially no perturbation in visual chromophore biosynthesis. This finding was significant because HPR does accumulate, albeit to a limited extent, within RPE tissue. Nevertheless, the therapeutic effect of HPR on halting the accumulation of A2E in abca-4−/− mice was only observed following a more prolonged treatment period. For example, abca-4−/− mice receiving 10 mg/kg HPR daily for 28 days accumulated ˜50% less A2E compared to littermates which received only DMSO (FIG. 10F). Interestingly, the level of RBP-retinol in the circulation was also reduced by ˜50% during this treatment period. Thus the reduction of A2E is related to reduced availability of retinol for uptake by the RPE.

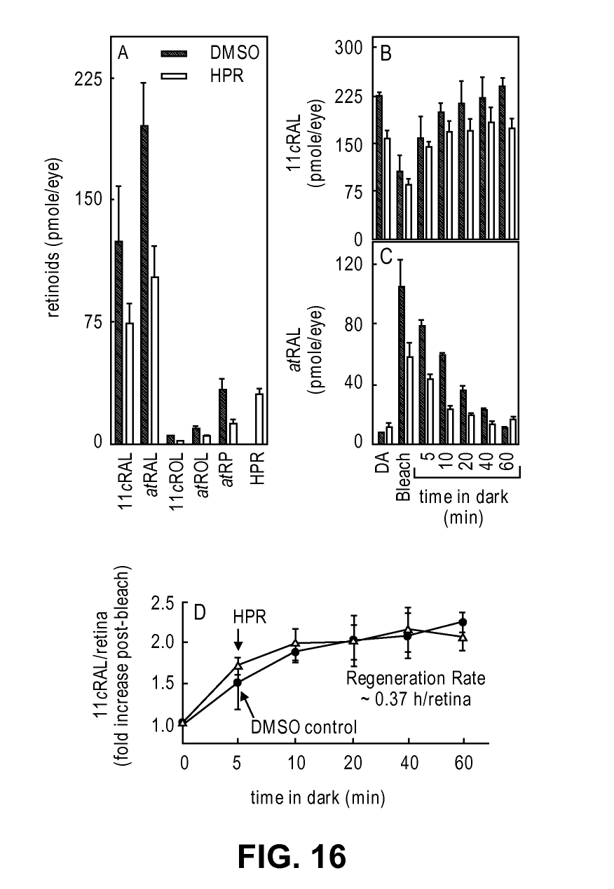

Both steady-state retinoid levels and rates of visual chromophore regeneration were evaluated in abca-4−/− mice following a 28-day treatment period with 10 mg/kg HPR. Light-adapted levels of all visual cycle retinoids were reduced by ˜50% compared to control animals receiving only DMSO (FIG. 16A). Although HPR was present within RPE tissue (˜10 μM), the rate of rhodopsin regeneration (FIG. 16B) and removal of bleached photoproduct (FIG. 16C) were not affected. The calculated time constant for regeneration of visual chromophore was nearly identical for both DMSO- and HPR-treated mice (0.37 h time constant to fully regenerate visual chromophore, FIG. 16D).

These data demonstrate that therapeutic doses of HPR do not affect the rate of visual chromophore biosynthesis. Thus, HPR does not interact with enzymes of the visual cycle. The observed reduction in A2E levels arises from lower steady-state levels of ocular retinoids which is due to reduced levels of RBP-retinol in the circulation. The relationship between HPR, serum retinol, ocular retinoids and A2E is shown in FIG. 12. This analysis reveals that HPR dose-dependent reductions in serum retinol produce commensurate reductions in ocular retinoids and A2E.

Latent Effects of HPR on Arresting A2E Accumulation

One feature identified during the HPR trials was a therapeutic latency effect following withdrawal of HPR. In these studies, chronic treatment of abca-4−/− mice (10 mg HPR/kg, i.p.) was stopped after 28 days. A2E levels were then measured 12, 28 and 42 days following the final HPR dose. The A2E levels remained persistently low for several weeks compared to untreated, age-matched control mice. This effect was not observed in animals treated with 13-cis retinoic acid, a result that may be due to the capacity of RPE cells to adapt to, and maintain, low steady-state retinoid levels. Further, photoreceptor function and ocular retinoid levels quickly returned to baseline values following withdrawal of 13-cis retinoic acid. These findings indicate that high steady-state levels of competitive inhibitors such as 13-cis retinoic acid must be maintained for therapeutic efficacy.

The Effect of HPR on Electrophysiology of the Retina

A prominent electrophysiological phenotype manifest by abca-4−/− mice is delayed-dark adaptation. Humans with mutations in the ABCA4 gene and those suffering from AMD also experience delayed-dark adaptation. This effect may be due to transient increases in pseudophotoproducts within rod photoreceptors. Under normal physiological conditions, photobleaching of rhodopsin generates an all-trans retinal-opsin conjugate (known as Metarhodopsin II, MII) within the lumen of rod discs. MII activates phototransduction machinery and is then quickly deactivated in order to restore dark sensitivity to the rod cell. Following deactivation of MII the chemical bond which couples all-trans retinal to opsin is hydrolyzed releasing all-trans retinal, which is subsequently removed from the disc lumen. In certain situations, MII is deactivated but the all-trans retinal-opsin bond remains intact. This species, referred to as a pseudophotoproduct, continues to mildly stimulate phototransduction machinery and produces a background “noise” which prolongs the time required for the rod cell to regain dark sensitivity.

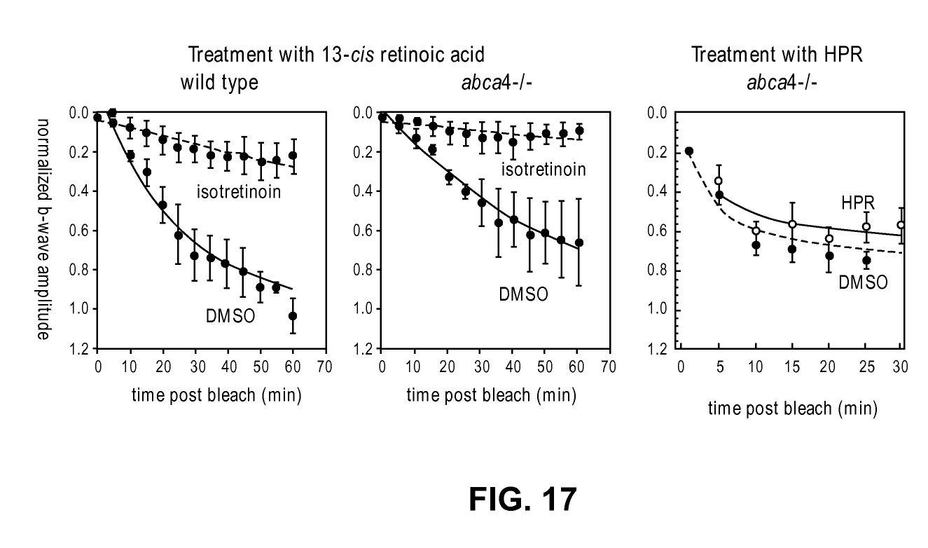

Delayed-dark adaptation can be further exacerbated by compounds which reduce the rate of rhodopsin regeneration (e.g., 13-cis retinoic acid). Although compounds such as HPR, which reduce total ocular retinoid levels, may also contribute to delayed-dark adaptation, the effect is less pronounced. This point was illustrated in studies which examined the effects of chronic (1 month) 13-cis retinoic acid or HPR administration on electrophysiology of the retina. The data (FIG. 17) reveal that 13-cis retinoic acid, which achieves a ˜50% reduction in A2E at 40 mg/kg, produces a considerable delay in the time required to regain dark sensitivity in wild-type and abca-4−/− mice. Following exposure to a light source which bleaches approximately 40% of the visual chromophore, wild-type mice require ˜1 hour to regain dark sensitivity (1.0 value on the y-axis); untreated abca-4−/− mice require ˜3-4 hours. 13-cis retinoic acid treatment prolongs the time required to regain dark sensitivity to several hours. In contrast, HPR, which achieves the same level of therapeutic efficacy at 10 mg/kg, does not significantly worsen the inherent delayed dark adaptation phenotype present in abca-4−/− mice (right panel). This finding is relevant for human patients affected with AMD. Like the abca-4−/− mice, these patients massively accumulate retinal fluorophores and also exhibit delayed-dark adaptation. It is better to treat such patients with compounds, such as HPR, that do not further compromise vision in dim light environments.

### Synthesis of the Compounds of Formula (I)

Compounds of Formula (I) may be synthesized using standard synthetic techniques known to those of skill in the art or using methods known in the art in combination with methods described herein. See, e.g., U.S. Patent Application Publication 2004/0102650; Um, S. J., et al., Chem. Pharm. Bull., 52:501-506 (2004). In addition, several of the compounds of Formula (I), such as fenretinide, may be purchased from various commercial suppliers. As a further guide the following synthetic methods may also be utilized.

Formation of Covalent Linkages by Reaction of an Electrophile with a Nucleophile

Selected examples of covalent linkages and precursor functional groups which yield them are given in the Table entitled “Examples of Covalent Linkages and Precursors Thereof” Precursor functional groups are shown as electrophilic groups and nucleophilic groups. The functional group on the organic substance may be attached directly, or attached via any useful spacer or linker as defined below.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

In general, carbon electrophiles are susceptible to attack by complementary nucleophiles, including carbon nucleophiles, wherein an attacking nucleophile brings an electron pair to the carbon electrophile in order to form a new bond between the nucleophile and the carbon electrophile.

Suitable carbon nucleophiles include, but are not limited to alkyl, alkenyl, aryl and alkynyl Grignard, organolithium, organozinc, alkyl-, alkenyl, aryl- and alkynyl-tin reagents (organostannanes), alkyl-, alkenyl-, aryl- and alkynyl-borane reagents (organoboranes and organoboronates); these carbon nucleophiles have the advantage of being kinetically stable in water or polar organic solvents. Other carbon nucleophiles include phosphorus ylids, enol and enolate reagents; these carbon nucleophiles have the advantage of being relatively easy to generate from precursors well known to those skilled in the art of synthetic organic chemistry. Carbon nucleophiles, when used in conjunction with carbon electrophiles, engender new carbon-carbon bonds between the carbon nucleophile and carbon electrophile.

Non-carbon nucleophiles suitable for coupling to carbon electrophiles include but are not limited to primary and secondary amines, thiols, thiolates, and thioethers, alcohols, alkoxides, azides, semicarbazides, and the like. These non-carbon nucleophiles, when used in conjunction with carbon electrophiles, typically generate heteroatom linkages (C—X—C), wherein X is a hetereoatom, e.g., oxygen or nitrogen.

### Use of Protecting Groups

The term “protecting group” refers to chemical moieties that block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester derivatives as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

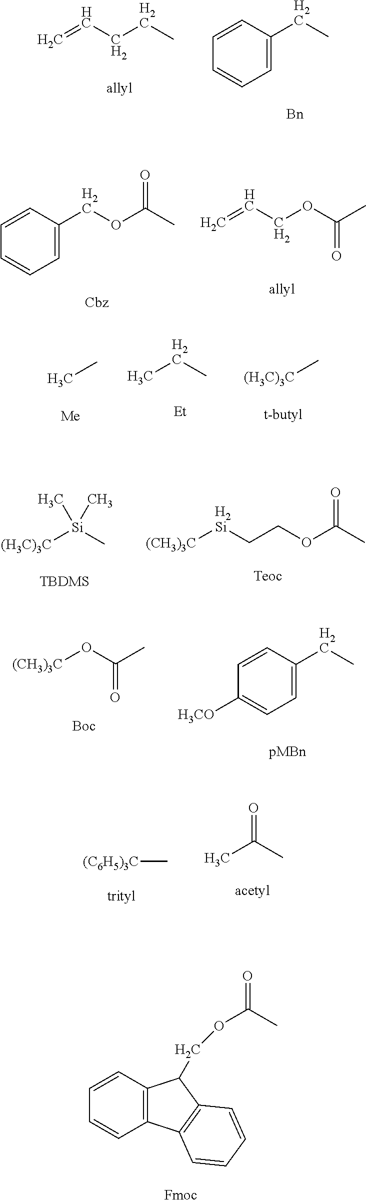

Other protecting groups are described in Greene and Wuts, Protective Groups in Organic Synthesis, 3rd Ed., John Wiley & Sons, New York, N.Y., 1999, which is incorporated herein by reference in its entirety.

### ILLUSTRATIVE EXAMPLES

The following examples provide illustrative methods for testing the effectiveness and safety of the compounds of Formula (I). These examples are provided for illustrative purposes only and not to limit the scope of the claims provided herein.

### Human Studies

Detection of Macular or Retinal Degeneration

Identification of abnormal blood vessels in the eye can be done with an angiogram. This identification can help determine which patients are candidates for the use of a candidate substance or other treatment method to hinder or prevent further vision loss. Angiograms can also be useful for follow-up of treatment as well as for future evaluation of any new vessel growth.

A fluorescein angiogram (fluorescein angiography, fluorescein angioscopy) is a technique for the visualization of choroidal and retinal circulation at the back of the eye. Fluorescein dye is injected intravenously followed by multiframe photography (angiography), ophthalmoscopic evaluation (angioscopy), or by a Heidelberg retina angiograph (a confocal scanning laser system). Additionally, the retina can be examined by OCT, a non-invasive way to obtain high-resolution cross-sectional images of the retina. Fluorescein angiograms are used in the evaluation of a wide range of retinal and choroidal diseases through the analysis of leakage or possible damage to the blood vessels that feed the retina. It has also been used to evaluate abnormalities of the optic nerve and iris by Berkow et al., Am. J. Ophthalmol. 97:143-7 (1984).

Similarly, angiograms using indocyanine green can be used for the visualization circulation at the back of the eye. Wherein fluorescein is more efficient for studying retinal circulation, indocyanine is better for observing the deeper choroidal blood vessel layer. The use of indocyanine angiography is helpful when neovascularization may not be observed with fluorescein dye alone.

Appropriate human doses for compounds having the structure of Formula (I) will be determined using a standard dose escalation study. However, some guidance is available from studies on the use of such compounds in the treatment of cancer. For example, a 4800 mg/m² dose of fenretinide, which is a compound having the structure of Formula (I), has been administered to patients with a variety of cancers. Such doses were administered three times daily and observed toxicities were minimal. However, the recommended dose for such patients was 900 mg/m² based on an observed ceiling on achievable plasma levels. In addition, the bioavailability of fenretinide is increased with meals, with the plasma concentration being three times greater after high fat meals than after carbohydrate meals.

### Example 1

### Testing for the Efficacy of Compounds of Formula (I) to Treat Macular Degeneration

For pre-testing, all human patients undergo a routine ophthalmologic examination including fluorescein angiography, measurement of visual acuity, electrophysiologic parameters and biochemical and rheologic parameters. Inclusion criteria are as follows: visual acuity between 20/160 and 20/32 in at least one eye and signs of AMD such as drusen, areolar atrophy, pigment clumping, pigment epithelium detachment, or subretinal neovascularization. Patients that are pregnant or actively breast-feeding children are excluded from the study.

Two hundred human patients diagnosed with macular degeneration, or who have progressive formations of A2E, lipofuscin, or drusen in their eyes are divided into a control group of about 100 patients and an experimental group of 100 patients. Fenretinide is administered to the experimental group on a daily basis. A placebo is administered to the control group in the same regime as fenretinide is administered to the experimental group.

Administration of fenretinide or placebo to a patient can be either orally or parenterally administered at amounts effective to inhibit the development or reoccurrence of macular degeneration. Effective dosage amounts are in the range of from about 1-4000 mg/m² up to three times a day.

One method for measuring progression of macular degeneration in both control and experimental groups is the best corrected visual acuity as measured by Early Treatment Diabetic Retinopathy Study (ETDRS) charts (Lighthouse, Long Island, N.Y.) using line assessment and the forced choice method (Ferris et al. Am J Ophthalmol, 94:97-98 (1982)). Visual acuity is recorded in logMAR. The change of one line on the ETDRS chart is equivalent to 0.1 logMAR. Further typical methods for measuring progression of macular degeneration in both control and experimental groups include use of visual field examinations, including but not limited to a Humphrey visual field examination and microperimetry (using, e.g., Micro Perimeter MP-1 from NIDEK), and measuring/monitoring the autofluorescence or absorption spectra of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine in the eye of the patient. Autofluorescence is measured using a variety of equipment, including but not limited to a confocal scanning laser ophthalmoscope. See Bindewald, et al., Am. J. Ophthalmol., 137:556-8 (2004).

Additional methods for measuring progression of macular degeneration in both control and experimental groups include taking fundus photographs, observing changes in autofluorescence over time using a Heidelberg retina angiograph (or alternatively, techniques described in M. Hammer, et al. Ophthalmologe 2004 Apr. 7 [Epub ahead of patent]), and taking fluorescein angiograms at baseline, three, six, nine and twelve months at follow-up visits. Documentation of morphologic changes include changes in (a) drusen size, character, and distribution; (b) development and progression of choroidal neovascularization; (c) other interval fundus changes or abnormalities; (d) reading speed and/or reading acuity; (e) scotoma size; or (f) the size and number of the geographic atrophy lesions. In addition, Amsler Grid Test and color testing are optionally administered.

To assess statistically visual improvement during drug administration, examiners use the ETDRS (LogMAR) chart and a standardized refraction and visual acuity protocol. Evaluation of the mean ETDRS (LogMAR) best corrected visual acuity (BCVA) from baseline through the available post-treatment interval visits can aid in determining statistical visual improvement.

To assess the ANOVA (analysis of variance between groups) between the control and experimental group, the mean changes in ETDRS (LogMAR) visual acuity from baseline through the available post-treatment interval visits are compared using two-group ANOVA with repeated measures analysis with unstructured covariance using SAS/STAT Software (SAS Institutes Inc, Cary, N.C.).

Toxicity evaluation after the commencement of the study include check ups every three months during the subsequent year, every four months the year after and subsequently every six months. Plasma levels of fenretinide, its metabolite N-(4-methoxyphenyl)-retinamide, serum retinol and/or RBP can also be assessed during these visits. The toxicity evaluation includes patients using fenretinide as well as the patients in the control group.

### Example 2

### Testing for the Efficacy of Compounds of Formula (I) to Reduce A2E Production

The same protocol design, including pre-testing, administration, dosing and toxicity evaluation protocols, that are described in Example 1 are also used to test for the efficacy of compounds of Formula (I) in reducing or otherwise limiting the production of A2E in the eye of a patient.

Methods for measuring or monitoring formation of A2E include the use of autofluorescence measurements of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine in the eye of the patient. Autofluorescence is measured using a variety of equipment, including but not limited to a confocal scanning laser ophthalmoscope, see Bindewald, et al., Am. J. Ophthalmol., 137:556-8 (2004), or the autofluorescence or absorption spectra measurement techniques noted in Example 1. Other tests that can be used as surrogate markers for the efficacy of a particular treatment include the use of visual acuity and visual field examinations (including, by way of example, microperimetry), reading speed and/or reading acuity examinations, measurements on the size and number of scotoma and/or geographic atrophic lesions, as described in Example 1. The statistical analyses described in Example 1 is employed.

### Example 3

### Testing for the Efficacy of Compounds of Formula (I) to Reduce Lipofuscin Production

The same protocol design, including pre-testing, administration, dosing and toxicity evaluation protocols, that are described in Example 1 are also used to test for the efficacy of compounds of Formula (I) in reducing or otherwise limiting the production of lipofuscin in the eye of a patient. The statistical analyses described in Example 1 may also be employed.

Tests that can be used as surrogate markers for the efficacy of a particular treatment include the use of visual acuity and visual field examinations (including, by way of example, microperimetry), reading speed and/or reading acuity examinations, measurements on the size and number of scotoma and/or geographic atrophic lesions, and the measuring/monitoring of autofluorescence of certain compounds in the eye of the patient, as described in Example 1.

### Example 4

### Testing for the Efficacy of Compounds of Formula (I) to Reduce Drusen Production

The same protocol design, including pre-testing, administration, dosing and toxicity evaluation protocols, that are described in Example 1 are also used to test for the efficacy of compounds of Formula (I) in reducing or otherwise limiting the production or formation of drusen in the eye of a patient. The statistical analyses described in Example 1 may also be employed.

Methods for measuring progressive formations of drusen in both control and experimental groups include taking fundus photographs and fluorescein angiograms at baseline, three, six, nine and twelve months at follow-up visits. Documentation of morphologic changes may include changes in (a) drusen size, character, and distribution (b) development and progression of choroidal neovascularization and (c) other interval fundus changes or abnormalities. Other tests that can be used as surrogate markers for the efficacy of a particular treatment include the use of visual acuity and visual field examinations (including, by way of example, microperimetry), reading speed and/or reading acuity examinations, measurements on the size and number of scotoma and/or geographic atrophic lesions, and the measuring/monitoring of autofluorescence of certain compounds in the eye of the patient, as described in Example 1.

### Example 5

### Genetic Testing for Macular Dystrophies

Defects in the human ABCA4 gene are thought to be associated with five distinct retinal phenotypes including Stargardt Disease, cone-rod dystrophy, age-related macular degeneration (both dry form and wet form) and retinitis pigmentosa. See e.g., Allikmets et al., Science, 277:1805-07 (1997); Lewis et al., Am. J. Hum. Genet., 64:422-34 (1999); Stone et al., Nature Genetics, 20:328-29 (1998); Allikmets, Am. J. Hum. Gen., 67:793-799 (2000); Klevering, et al, Ophthalmology, 111:546-553 (2004). In addition, an autosomal dominant form of Stargardt Disease is caused by mutations in the ELOV4 gene. See Karan, et al., Proc. Natl. Acad. Sci. (2005). Patients can be diagnosed as having Stargardt Disease by any of the following assays:
A direct-sequencing mutation detection strategy which can involve sequencing all exons and flanking intron regions of ABCA4 or ELOV4 for sequence mutation(s);
Genomic Southern analysis;
Microarray assays that include all known ABCA4 or ELOV4 variants; and
Analysis by liquid chromatography tandem mass spectrometry coupled with immunocytochemical analysis using antibodies and Western analysis.

Fundus photographs, fluorescein anigograms, and scanning laser ophthalmoscope imaging along with the history of the patient and his or her family can anticipate and/or confirm diagnosis.

### Mice and Rat Studies

The optimal dose of compounds of Formula (I) to block formation of A2E in abca4⁻/⁻ mice can be determined using a standard dose escalation study. One illustrative approach, utilizing fenretinide, which is a compound having the structure of Formula (I) is presented below. However, similar approaches may be utilized for other compounds having the structure of Formula (I).

The effects of fenretinide on all-trans-retinal in retinas from light-adapted mice would preferably be determined at doses that bracket the human therapeutic dose. The preferred method includes treating mice with a single morning intraperitoneal dose. An increased frequency of injections may be required to maintain reduced levels of all-trans-retinal in the retina throughout the day.

ABCA4 Knockout Mice

ABCA4 encodes rim protein (RmP), an ATP-binding cassette (ABC) transporter in the outer-segment discs of rod and cone photoreceptors. The transported substrate for RmP is unknown. Mice generated with a knockout mutation in the abca4 gene, see Weng et al., Cell, 98:13-23 (1999), are useful for the study of RmP function as well as for an in vivo screening of the effectiveness for candidate substances. These animals manifest the complex ocular phenotype: (i) slow photoreceptor degeneration, (ii) delayed recovery of rod sensitivity following light exposure, (iii) elevated atRAL and reduced atROL in photoreceptor outer-segments following a photobleach, (iv) constitutively elevated phosphatidylethanolamine (PE) in outer-segments, and (v) accumulation of lipofuscin in RPE cells. See Weng et al., Cell, 98:13-23 (1999).

Rates of photoreceptor degeneration can be monitored in treated and untreated wild-type and abca4⁻/⁻ mice by two techniques. One is the study of mice at different times by ERG analysis and is adopted from a clinical diagnostic procedure. See Weng et al., Cell, 98:13-23 (1999). An electrode is placed on the corneal surface of an anesthetized mouse and the electrical response to a light flash is recorded from the retina. Amplitude of the α-wave, which results from light-induced hyperpolarization of photoreceptors, is a sensitive indicator of photoreceptor degeneration. See Kedzierski et al., Invest. Ophthalmol. Vis. Sci., 38:498-509 (1997). ERGs are done on live animals. The same mouse can therefore be analyzed repeatedly during a time-course study. The definitive technique for quantitating photoreceptor degeneration is histological analysis of retinal sections. The number of photoreceptors remaining in the retina at each time point will be determined by counting the rows of photoreceptor nuclei in the outer nuclear layer.

Tissue Extraction

Eye samples were thawed on ice in 1 ml of PBS, pH 7.2 and homogenized by hand using a Duall glass-glass homogenizer. The sample was further homogenized following the addition of 1 ml chloroform/methanol (2:1, v/v). The sample was transferred to a boroscilicate tube and lipids were extracted into 4 mls of chloroform. The organic extract was washed with 3 mls PBS, pH 7.2 and the samples were then centrifuged at 3,000×g, 10 min. The choloroform phase was decanted and the aqueous phase was re-extracted with another 4 mls of chloroform. Following centrifugation, the chloroform phases were combined and the samples were taken to dryness under nitrogen gas. Samples residues were resuspended in 100 μl hexane and analyzed by HPLC as described below.

HPLC Analysis

Chromatographic separations were achieved on an Agilent Zorbax Rx-Sil Column (5 μm, 4.6×250 mm) using an Agilent 1100 series liquid chromatograph equipped with fluorescence and diode array detectors. The mobile phase (hexane/2-propanol/ethanol/25 mM KH₂PO₄, pH 7.0/acetic acid; 485/376/100/50/0.275, v/v) was delivered at 1 ml/min. Sample peak identification was made by comparison to retention time and absorbance spectra of authentic standards. Data are reported as peak fluorescence (L.U.) obtained from the fluorescence detector.

### Example 6

### Effect of Fenretinide on A2E Accumulation

Administration of fenretinide to an experimental group of mice and administration of DMSO alone to a control group of mice is performed and assayed for accumulation of A2E. The experimental group is given 2.5 to 20 mg/kg of fenretinide per day in 10 to 25 μl of DMSO. Higher dosages are tested if no effect is seen with the highest dose of 50 mg/kg. The control group is given 10 to 25 μl injections of DMSO alone. Mice are administered either experimental or control substances by intraperitoneal (i.p.) injection for various experimental time periods not to exceed one month.

To assay for the accumulation of A2E in abca4⁻/⁻ mice RPE, 2.5 to 20 mg/kg of fenretinide is provided by i.p. injection per day to 2-month old abca4⁻/⁻ mice. After 1 month, both experimental and control mice are killed and the levels of A2E in the RPE are determined by HPLC. In addition, the autofluorescence or absorption spectra of N-retinylidene-phosphatidylethanolamine, dihydro-N-retinylidene-N-retinyl-phosphatidylethanolamine, N-retinylidene-N-retinyl-phosphatidylethanolamine, dihydro-N-retinylidene-N-retinyl-ethanolamine, and/or N-retinylidene-phosphatidylethanolamine may be monitored using a UV/Vis spectrophotometer.

### Example 7

### Effect of Fenretinide on Lipofuscin Accumulation

Administration of fenretinide to an experimental group of mice and administration of DMSO alone to a control group of mice is performed and assayed for the accumulation of lipofuscin. The experimental group is given 2.5 to 20 mg/kg of fenretinide per day in 10 to 25 μl of DMSO. Higher dosages are tested if no effect is seen with the highest dose of 50 mg/kg. The control group are given 10 to 25 μl injections of DMSO alone. Mice are administered either experimental or control substances by i.p. injection for various experimental time periods not to exceed one month. Alternatively, mice can be implanted with a pump which delivers either experimental or control substances at a rate of 0.25 μl/hr for various experimental time periods not to exceed one month.

To assay for the effects of fenretinide on the formation of lipofuscin in fenretinide treated and untreated abca4⁻/⁻ mice, eyes can be examined by electron or fluorescence microscopy.

### Example 8

### Effect of Fenretinide on Rod Cell Death or Rod Functional Impairment

Administration of fenretinide to an experimental group of mice and administration of DMSO alone to a control group of mice is performed and assayed for the effects of fenretinide on rod cell death or rod functional impairment. The experimental group is given 2.5 to 20 mg/kg of fenretinide per day in 10 to 25 μl of DMSO. Higher dosages are tested if no effect is seen with the highest dose of 50 mg/kg. The control group is given 10 to 25 μl injections of DMSO alone. Mice are administered either experimental or control substances by i.p. injection for various experimental time periods not to exceed one month. Alternatively, mice can be implanted with a pump which delivers either experimental or control substances at a rate of 0.25 μl/hr for various experimental time periods not to exceed one month.

Mice that are treated to 2.5 to 20 mg/kg of fenretinide per day for approximately 8 weeks can be assayed for the effects of fenretinide on rod cell death or rod functional impairment by monitoring ERG recordings and performing retinal histology.

### Example 9

### Testing for Protection from Light Damage

The following study is adapted from Sieving, P. A., et al, Proc. Natl. Acad. Sci., 98:1835-40 (2001). For chronic light-exposure studies, Sprague-Dawley male 7-wk-old albino rats are housed in 12:12 h light/dark cycle of 5 lux fluorescent white light. Injections of 20-50 mg/kg fenretinide by i.p. injection in 0.18 ml DMSO are given three times daily to chronic rats for 8 wk. Controls receive 0.18 ml DMSO by i.p. injection. Rats are killed 2 d after final injections. Higher dosages are tested if no effect is seen with the highest dose of 50 mg/kg.

For acute light-exposure studies, rats are dark-adapted overnight and given a single i.p. injection of fenretinide 20-50 mg/kg in 0.18 ml DMSO under dim red light and kept in darkness for 1 h before being exposed to the bleaching light before ERG measurements. Rats exposed to 2,000 lux white fluorescent light for 48 h. ERGs are recorded 7 d later, and histology is performed immediately.

Rats are euthanized and eyes are removed. Column cell counts of outer nuclear layer thickness and rod outer segment (ROS) length are measured every 200 μm across both hemispheres, and the numbers are averaged to obtain a measure of cellular changes across the entire retina. ERGs are recorded from chronic rats at 4 and 8 wks of treatment. In acute rodents, rod recovery from bleaching light is tracked by dark-adapted ERGs by using stimuli that elicit no cone contribution. Cone recovery is tracked with photopic ERGs. Prior to ERGs, animals are prepared in dim red light and anaesthetized. Pupils are dilated and ERGs are recorded from both eyes simultaneously by using gold-wire corneal loops.

### Example 10

### Combination Therapy Involving Fenretinide and Accutane

Mice and/or rats are tested in the manner described in Examples 6-9, but with an additional two arms. In one of the additional arms, groups of mice and/or rats are treated with increasing doses of Accutane, from 5 mg/kg per day to 50 mg/kg per day. In the second additional arm, groups of mice and/or rats are treated with a combination of 20 mg/kg per day of fenretinide and increasing doses of Accutane, from 5 mg/kg per day to 50 mg/kg per day. The benefits of the combination therapy are assayed as described in Examples 6-9.

### Example 11

### Efficacy of Fenretinide on the Accumulation of Lipofuscin (and/or A2E) in abca4 Null Mutant Mice: Phase I—Dose Response and Effect on Serum Retinol

The effect of HPR on reducing serum retinol in animals and human subjects led us to explore the possibility that reductions in lipofuscin and the toxic bis-retinoid conjugate, A2E, may also be realized. The rationale for this approach is based upon two independent lines of scientific evidence: 1) reduction in ocular vitamin A concentration via inhibition of a known visual cycle enzyme (11-cis retinol dehydrogenase) results in profound reductions in lipofuscin and A2E; 2) animals maintained on a vitamin A deficient diet demonstrate dramatic reductions in lipofuscin accumulation. Thus, the objective for this example was to examine the effect of HPR in an animal model which demonstrates massive accumulation of lipofuscin and A2E in ocular tissue, the abca4 null mutant mouse.

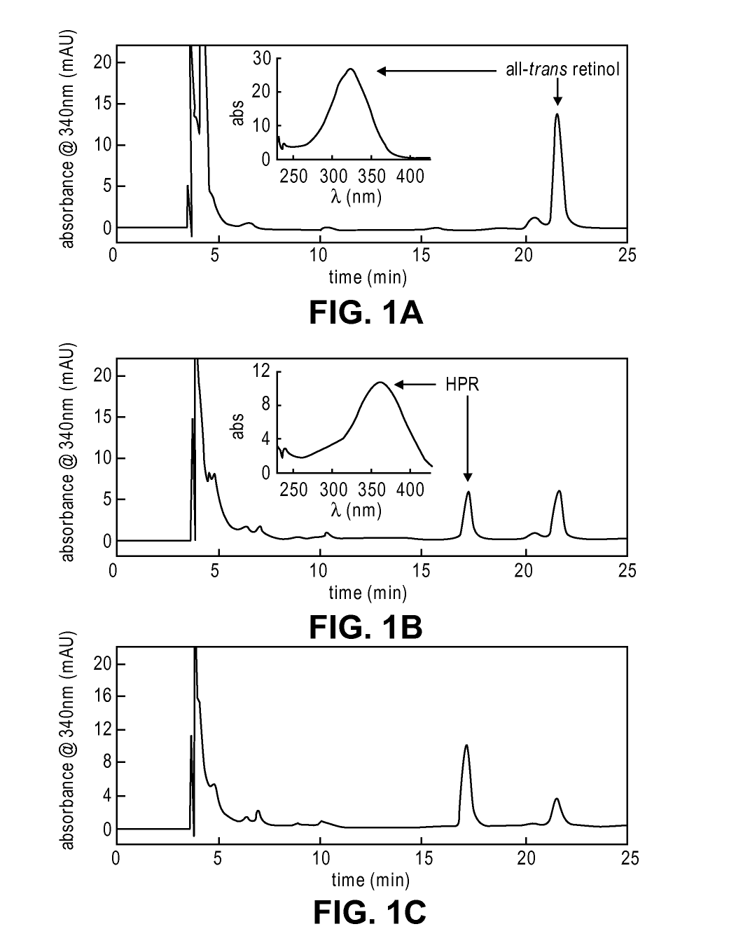

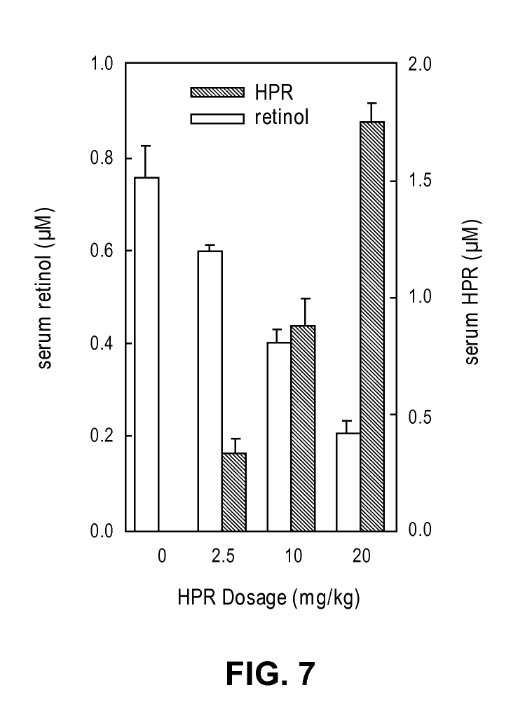

Initial studies began by examining the effect of HPR on serum retinol. Animals were divided into three groups and given either DMSO, 10 mg/kg HPR, or 20 mg/kg HPR for 14 days. At the end of the study period, blood was collected from the animals, sera were prepared and an acetonitrile extract of the serum was analyzed by reverse phase LC/MS. UV-visible spectral and mass/charge analyses were performed to confirm the identity of the eluted peaks. Sample chromatograms obtained from these analyses are shown: FIG. 1a.—extract from an abca4 null mutant mouse receiving HPR vehicle, DMSO; FIG. 1b.—10 mg/kg HPR; FIG. 1c.—20 mg/kg HPR. The data clearly show a dose-dependent reduction in serum retinol. Quantitative data indicate that at 10 mg/kg HPR, all-trans retinol is decreased 40%, see FIG. 7. For 20 mg/kg HPR, serum retinol is decreased 72%, see FIG. 7. The steady state concentrations of retinol and HPR in serum (at 20 mg/kg HPR) were determined to be 2.11 μM and 1.75 μM, respectively.

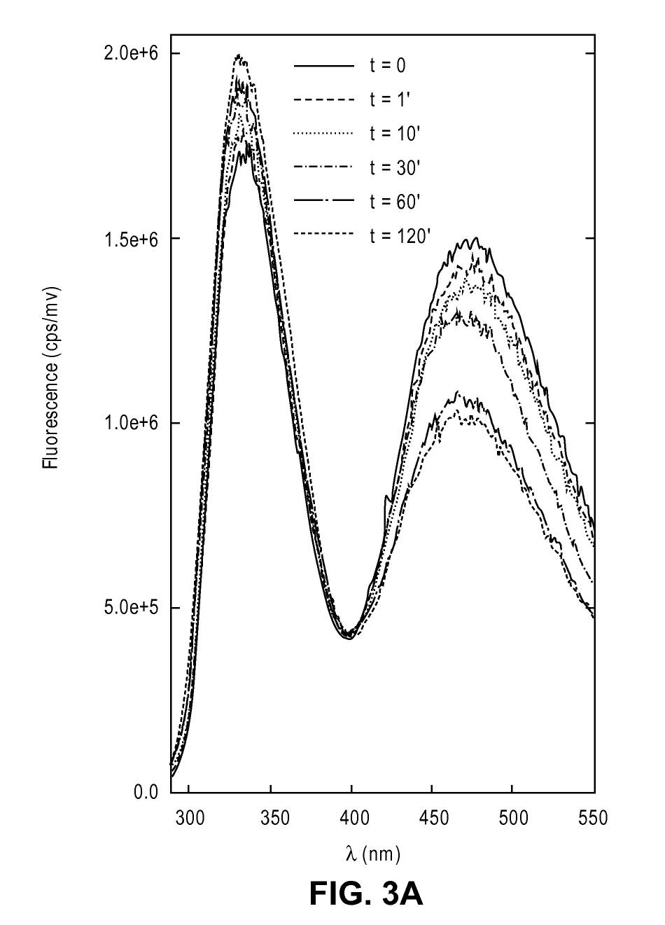

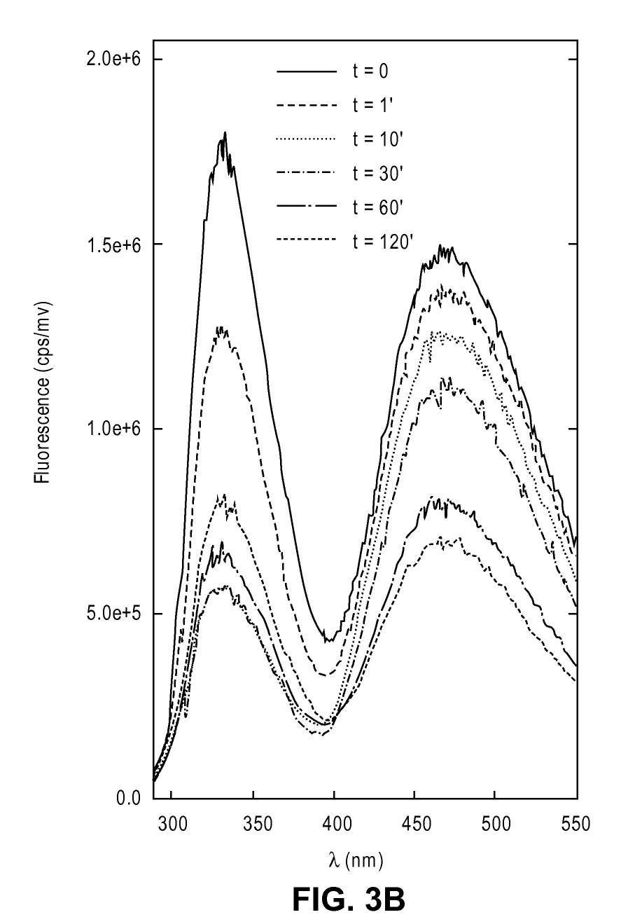

Based upon these findings, we sought to further explore the mechanism(s) of retinol reduction during HPR treatment. A tenable hypothesis is that HPR may displace retinol by competing at the retinol binding site on RBP. Like retinol, HPR will absorb (quench) light energy in the region of protein fluorescence; however, unlike retinol, HPR does not emit fluorescence. Therefore, one can measure displacement of retinol from the RBP holoprotein by observing decreases in both protein (340 nm) and retinol (470 nm) fluorescence. We performed a competition binding assay using RBP-retinol/HPR concentrations which were similar to those determined from the 14 day trial at 20 mg/kg HPR described above. Data obtained from these analyses reveal that HPR efficiently displaces retinol from the RBP-retinol holoprotein at physiological temperature, see FIG. 3b. The competitive binding of HPR to RBP was dose-dependent and saturable. In the control assays, decreases in retinol fluorescence were associated with concomitant increases in protein fluorescence, see FIG. 3a. This effect was determined to be due to temperature effects as the dissociation constant of RBP-retinol increases (decreased affinity) with increased time at 37 C. In summary, these data suggest that a molar equivalent of HPR, relative to RBP holoprotein (e.g., 1.0 μM), will displace retinol from RBP in vivo. Increases of HPR beyond equimolar amounts relative to RBP holoprotein (e.g., 2.0 μM HPR to 1.0 μM RBP) will produce a population of RBP which is largely associated with HPR.

Administration of an agent or agents that lower the levels of serum retinol in a patient without modulating at least one enzyme in the visual cycle is expected to provide a treatment for macular and/or retinal dystrophies and degenerations or the symptoms associated thereof. Assays, such as those described herein, may be used to select further agents possessing this action, including agents selected from compounds having the structure of Formula (I) as well as other agents. Putative lead compounds include other agents known or demonstrated to effect the serum level of retinol.

### Example 12

### Efficacy of Fenretinide on the Accumulation of Lipofuscin (and/or A2E) in abca4 Null Mutant Mice: Phase II—Chronic Treatment of abca4 Null Mutant Mice

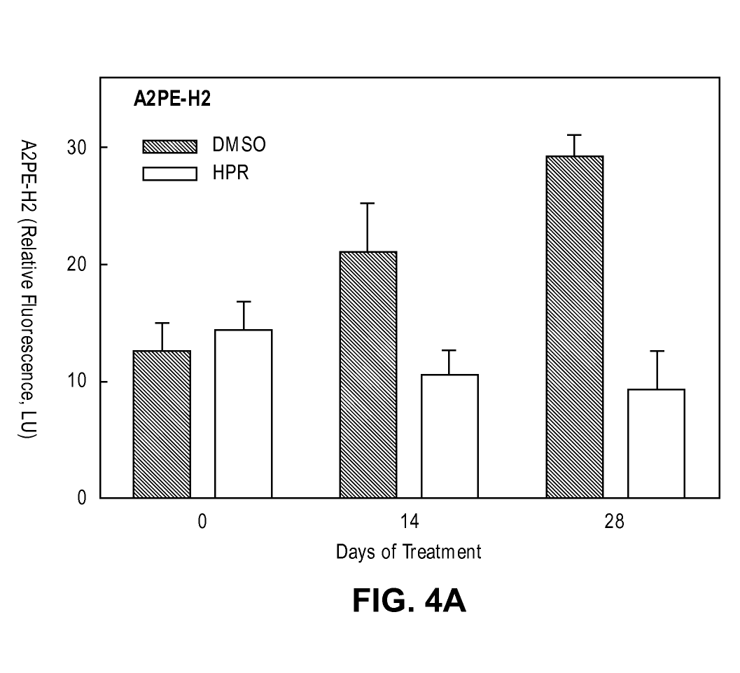

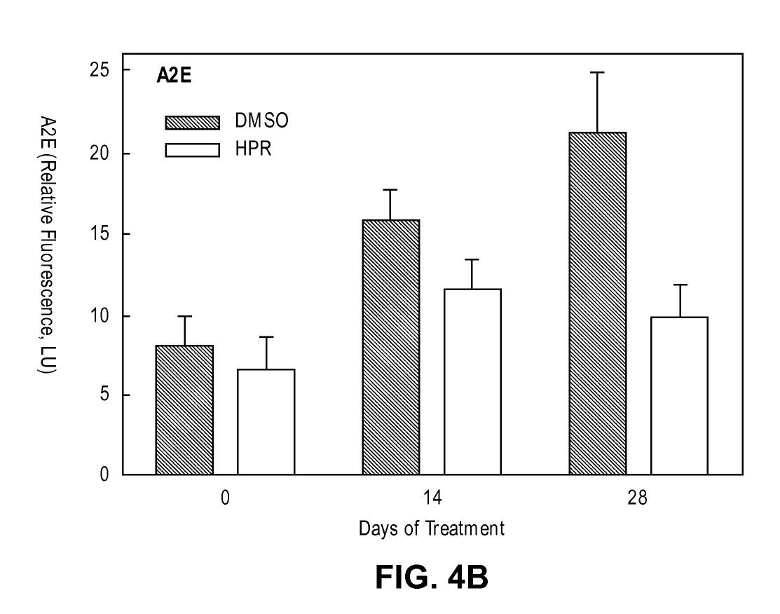

We initiated a one-month study to evaluate the effects of HPR on reduction of A2E and A2E precursors in abca4 null mutant mice. HPR was administered in DMSO (20 mg/kg, ip) to abca4 null mutant mice (BL6/129, aged 2 months) daily for a period of 28 days. Control age/strain matched mice received only the DMSO vehicle. Mice were sampled at 0, 14, and 28 days (n=3 per group), the eyes were enucleated and chloroform-soluble constituents (lipids, retinoids and lipid-retinoid conjugates) were extracted. Mice were sacrificed by cervical dislocation, the eyes were enucleated and individually snap frozen in cryo vials. The sample extracts were then analyzed by HPLC with on-line fluorescence detection. Results from this study show remarkable, early reductions in the A2E precursor, A2PE-H2, see FIG. 4a, and subsequent reductions in A2E, see FIG. 4b. Quantitative analysis revealed a 70% reduction of A2PE-H2 and 55% reduction of A2E following 28 days of HPR treatment. A similar study may be undertaken to ascertain effects of HPR treatment on the electroretinographic and morphological phenotypes.

### Example 13

### Combination Therapy Involving Fenretinide and a Statin

Mice and/or rats are tested in the manner described in Examples 6-9, but with an additional two arms. In one of the additional arms, groups of mice and/or rats are treated with a suitable statin such as: Lipitor® (Atorvastatin), Mevacor® (Lovastatin), Pravachol® (Pravastatin sodium), Zocor™ (Simvastatin), Leschol (fluvastatin sodium) and the like with optimal dosage based on weight. In the second additional arm, groups of mice and/or rats are treated with a combination of 20 mg/kg per day of fenretinide and increasing doses of the statin used in the previous step. Suggested human dosages of such statins are for example: Lipitor® (Atorvastatin) 10-80 mg/day, Mevacor® (Lovastatin) 10-80 mg/day, Pravachol® (Pravastatin sodium) 10-40 mg/day, Zocor™ (Simvastatin) 5-80 mg/day, Leschol (fluvastatin sodium) 20-80 mg/day. Dosage of statins for mice and/or rat subjects should be calculated based on weight. The benefits of the combination therapy are assayed as described in Examples 6-9.

### Example 14

### Combination Therapy Involving Fenretinide, Vitamins and Minerals

Mice and/or rats are tested in the manner described in Example 13, but with selected vitamins and minerals. Administration of fenretinide in combination with vitamins and minerals can be either orally or parenterally administered at amounts effective to inhibit the development or reoccurrence of macular degeneration. Test dosages are initially in the range of about 20 mg/kg per day of fenretinide with 100-1000 mg vitamin C, 100-600 mg vitamin E, 10,000-40,000 IU vitamin A, 50-200 mg zinc and 1-5 mg copper for 15 to 20 weeks. The benefits of the combination therapy are assayed as described in Examples 6-9.

### Example 15

### Fluorescence Quenching Study of Transthyretin (TTR) Binding to Retinol Binding Protein (RBP)

Apo-RBP at 0.5 μM was incubated with 0, 0.25, 0.5, 1 and 2 μM of MPR in PBS at room temperature for 1 hour, respectively. As controls, the same concentration of Apo-RBP was also incubated with 1 μM of HPR or 1 μM of atROL. All mixtures contained 0.2% Ethanol (v/v). The emission spectra were measured between 290 nm to 550 nm with excitation wavelength at 280 nm and 3 nm bandpass.

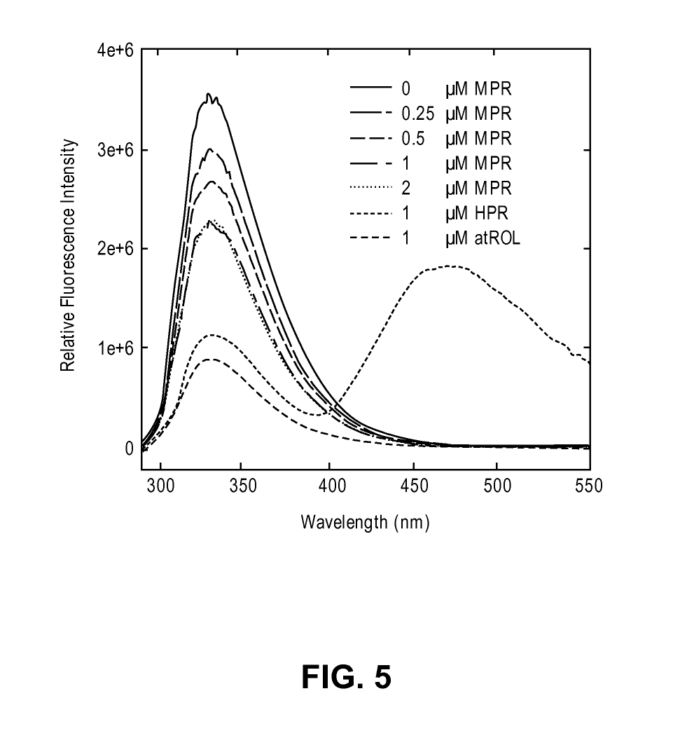

As shown in FIG. 5, MPR exhibited concentration-dependent quenching of RBP fluorescence, and the quenching saturated at 1 μM of MPR for 0.5 μM of RBP. Because the observed fluorescence quenching is likely due to fluorescence resonance energy transfer between protein aromatic residues and bound MPR molecule, MPR is proposed to bind to RBP. The degree of quenching by MPR is smaller than those by atROL and HPR, two other ligands that bind to RBP.

### Example 16

### Size Exclusion Study of TTR Binding to RBP

Apo-RBP at 10 μM was incubated with 50 μM of MPR in PBS at room temperature for 1 hour. 10 μM of TTR was then added to the solution, and the mixture was incubated for another hour at room temperature. 50 μA of the sample mixtures with and without TTR addition were analyzed by BioRad Bio-Sil SEC125 Gel Filtration Column (300×7.8 mm). In control experiments, atROL-RBP and atROL-RBP-TTR mixture were analyzed in the same manner.

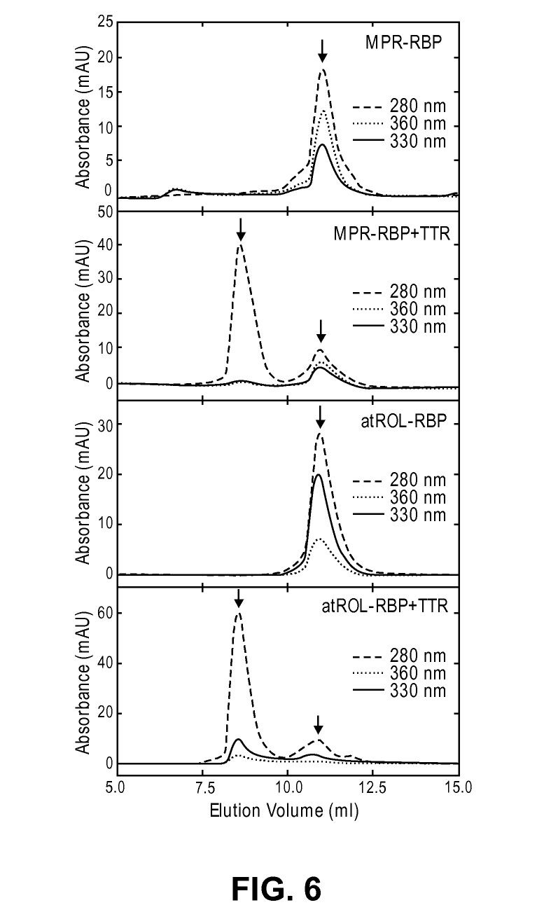

As shown in FIG. 6a, the MPR-RBP sample exhibited an RBP elution peak (at 11 ml) with strong absorbance at 360 nm, indicating RBP binds to MPR; after incubation with TTR, this 360 nm absorbance stayed with the RBP elution peak, while TTR elution peak (at 8.6 ml) did not contain any apparent 360 nm absorbance (see FIG. 6b), indicating MPR-RBP did not bind to TTR. In atROL-RBP control experiment, RBP elution peak showed strong 330 nm absorbance (see FIG. 6c); after incubation with TTR, more than half of this 330 nm absorbance shifted to TTR elution peak (see FIG. 6d), indicating atROL-RBP binds to TTR. Thus, MPR inhibits the binding of TTR to RBP.

### Example 17

### Analysis of Serum Retinol as a Function of HPR Concentration

ABCA4 null mutant mice were given the indicated dose of HPR in DMSO (i.p.) daily for 28 days (n=4 mice per dosage group). At the end of the study period, blood samples were taken and serum was prepared. Following acetonitrile precipitation of serum proteins, the concentrations of retinol and HPR were determined from the soluble phase by LC/MS (see FIG. 7). Identity of the eluted compounds was confirmed by UV-vis absorption spectroscopy and co-elution of sample peaks with authentic standards.

### Example 18

### Correlation of HPR Concentration to Reductions in Retinol, A2PE-H₂ and A2E in ABCA4 Null Mutant Mice

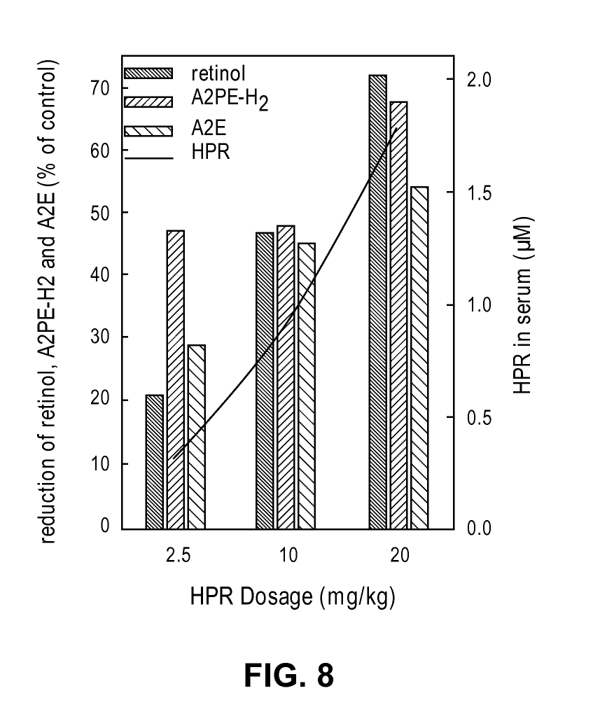

Group averages from the data shown in panels A-G of FIG. 10 in Example 19 (28 day time points) are plotted to illustrate the strong correlation between increases in serum HPR and decreases in serum retinol (see FIG. 8). Reductions in serum retinol are highly correlated with reductions in A2E and precursor compounds (A2PE-H₂). A pronounced reduction in A2PE-H₂ in the 2.5 mg/kg dosage group (˜47%) is observed when the serum retinol reduction is only 20%. The reason for this disproportionate reduction is related to the inherently lower ocular retinoid content in this group of 2-month old animals compared to the other groups. It is likely that if these animals had been maintained on the 2.5 mg/kg dose for a more prolonged period, a greater reduction in A2E would also be realized.

### Example 19

### Analysis of A2PE-H₂ and A2E Levels as a Function of HPR Dose and Treatment Period

Analysis of retinoid composition in light adapted DMSO- and HPR-treated mice (FIG. 9, panel A) shows approximately 50% reduction of visual cycle retinoids as a result of HPR treatment (10 mg/kg daily for 28 days). Panels B and C of FIG. 9 show that HPR does not affect regeneration of visual chromophore in these mice (panel B is visual chromophore biosynthesis, panel C is bleached chromophore recycling). Panels D-F of FIG. 9 are electrophysiological measurements of rod function (panel D), rod and cone function (panel E) and recovery from photobleaching (panel F). The only notable difference is delayed dark adaptation in the HPR-treated mice (panel F).

ABCA4 null mutant mice were given the indicated dose of HPR in DMSO or DMSO alone daily for 28 days (n=16 mice per treatment group). At study onset, mice in the 2.5 mg/kg group were 2 months of age, mice in the other treatment groups were 3 months of age. At the

indicated times, representative mice were taken from each group (n=4) for analysis of A2E precursor compounds (see FIG. 10, A2PE-H₂, panels A, C and E) and A2E (see FIG. 10, panels B, D and F). Eyes were enucleated, hemisected and lipid soluble components were extracted from the posterior pole by chloroform/methanol-water phase partitioning. Sample extracts were analyzed by LC. Identity of the eluted compounds was confirmed by UV-vis absorption spectroscopy and co-elution of sample peaks with authentic standards. Note: limitations in appropriately age and strain-matched mice in the 10 mg/kg group prevented analysis at the 14-day interval.

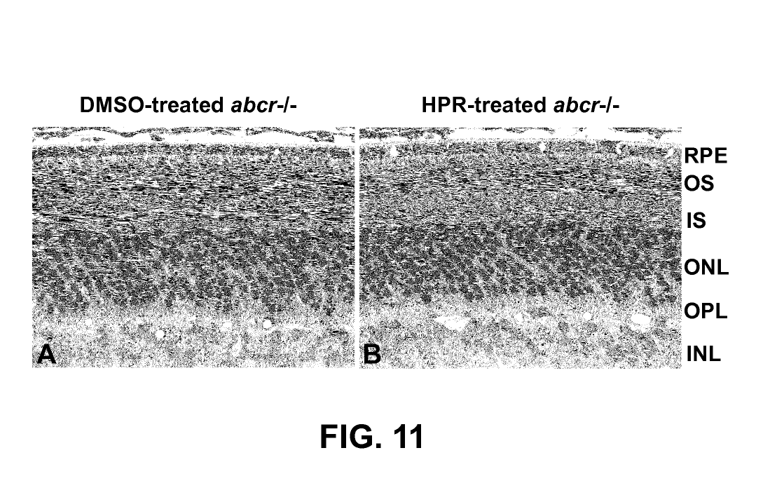

Panels G-I in FIG. 10 show morphological/histological evidence that HPR significantly reduces lipofuscin autofluorescence in the RPE of abcr null mutant mice (Stargardt's animal model). Treatment conditions are as described above. The level of autofluorescence in the HPR-treated animal is less than that of an age-matched wild-type animal. FIG. 11 shows light microscopy images of the retinas from DMSO- and HPR-treated animals show no aberrant morphology or compromise of the integrity in retinal cytostructure.

Accumulation of lipofuscin in the retinal pigment epithelium (RPE) is a common pathological feature observed in various degenerative diseases of the retina. A toxic vitamin A-based fluorophore (A2E) present within lipofuscin granules has been implicated in death of RPE and photoreceptor cells. In these experiments, we employed an animal model which manifests accelerated lipofuscin accumulation to evaluate the efficacy of a therapeutic approach based upon reduction of serum vitamin A (retinol). Fenretinide potently and reversibly reduces serum retinol. Administration of HPR to mice harboring a null mutation in the Stargardt's disease gene (ABCA4) produced profound reductions in serum retinol/retinol binding protein and arrested accumulation of A2E and lipofuscin autofluorescence in the RPE. Physiologically, HPR-induced reductions of visual chromophore were manifest as modest delays in dark adaptation; chromophore regeneration kinetics were normal. Importantly, specific intracellular effects of HPR on vitamin A esterification and chromophore mobilization were also identified. These findings demonstrate the vitamin A-dependent nature of A2E biosynthesis and validate a therapeutic approach which is readily transferable to human patients suffering from lipofuscin-based retinal diseases.

### Example 20

### Identification of Compounds that Bind to TTR and/or Inhibit Gene Expression of TTR

Purified TTR polypeptides comprising a glutathione-S-transferase protein and absorbed onto glutathione-derivatized wells of 96-well microtiter plates are contacted with test compounds from a small molecule library at pH 7.0 in a physiological buffer solution. Purified TTR polypeptides have been described in the art. See U.S. Patent App. No. 20020160394, herein incorporated by reference in its entirety. The test compounds may comprise a fluorescent tag. The samples are incubated for 5 minutes to one hour. Control samples are incubated in the absence of a test compound.

The buffer solution containing the test compounds is washed from the wells. Binding of a test compound to a TTR polypeptide is detected by fluorescence measurements of the contents of the wells. A test compound that increases the fluorescence in a well by at least 15% relative to fluorescence of a well in which a test compound is not incubated is identified as a compound which binds to a TTR polypeptide.

The identified test compound may be administered to a culture of human cells transfected with a TTR expression construct and incubated at 37° C. for 10 to 45 minutes. A culture of the same type of cells that have not been transfected is incubated for the same time without the test compound to provide a negative control.

RNA is then isolated from the two cultures as described in Chirgwin et al., Biochem. 18, 5294-99, 1979). Northern blots are prepared using 20 to 30 μg total RNA and hybridized with a ³²P-labeled TTR-specific probe. Probes for detecting TTR mRNA transcripts have been described previously. A test compound that decreases the TTR-specific signal relative to the signal obtained in the absence of the test compound is identified as an inhibitor of TTR gene expression.

### Example 21

### Identification of Compounds that Bind to RBP and/or Inhibit Gene Expression of RBP

Purified apo RBP are contacted with test compounds from a small molecule library at pH 7.0 in a physiological buffer solution. Purified apo RBP have been described in the art. See U.S. Patent App. No. 20030119715, herein incorporated by reference in its entirety. The test compounds may comprise a fluorescent tag. The samples are incubated for 5 minutes to one hour. Control samples are incubated in the absence of a test compound. Competition assays in the presence of holo RBP (RBP complexed with retinol) may also be performed.

The buffer solution containing the test compounds is washed from the wells. Binding of a test compound to apo RBP is detected by fluorescence measurements of the contents of the wells. A test compound that increases the fluorescence in a well by at least 15% relative to fluorescence of a well in which a test compound is not incubated is identified as a compound which binds to apo RBP.

The identified test compound may be administered to a culture of human cells transfected with an RBP expression construct and incubated at 37° C. for 10 to 45 minutes. A culture of the same type of cells that have not been transfected is incubated for the same time without the test compound to provide a negative control.

RNA is then isolated from the two cultures as described in Chirgwin et al., Biochem. 18, 5294-99, 1979). Northern blots are prepared using 20 to 30 μg total RNA and hybridized with a ³²P-labeled RBP-specific probe. A test compound that decreases the RBP-specific signal relative to the signal obtained in the absence of the test compound is identified as an inhibitor of RBP gene expression.

### Example 22

### Further Analysis of the Effect of HPR on Serum Retinol, Eyecup Retinoids, and A2E Levels

HPR Treatments

HPR was administered daily (1.5-15 μg/μl in 25 μl DMSO, i.p.) to ABCA4−/− mice for 28 days. Mice were 1-2 months of age at study onset and were either pigmented (129/SV) or albino (BALB/c) strains. Mice were raised under 12-hr cyclic light/dark (30-50 lux) during the treatment period and were anesthetized by i.p. injection of ketamine (200 mg/kg) plus xylazine (10 mg/kg) before death by cervical dislocation.

Analysis of Serum Retinol

Whole blood was collected from tail veins of HPR-treated mice 18 hrs. following the final HPR dose (i.e., at day 28). Serum was obtained from whole blood following centrifugation at 1,500×g, 10 min. Serum proteins were precipitated with the addition of an equivolume of ice-cold acetonitrile and centrifugation (10,000×g, 10 min). An aliquot was removed from the soluble phase and analyzed by HPLC using an Agilent 1100 series capillary liquid chromatograph equipped with a diode-array detector. Chromatography was performed on a Zorbax SB C18 5 μm column (150×0.5 mm) equilibrated with acetonitrile/water/glacial acetic acid (80:18:2, v/v) at a flow rate of 10 μl/min.

Extraction and Analysis of Retinoids and A2E

Steady-state levels of retinoids and A2E in eyecups of ABCA4−/− mice were determined following daily administration (28 days) of HPR (FIG. 12). Mice were sacrificed, the eyes enucleated, and the posterior portion of each eye was used for extraction of retinoids or A2E. Methodologies used for extraction of retinoids and A2E from eye tissue and HPLC analysis techniques have been described. See, e.g., Mata N L, Weng J, Travis G H. Biosynthesis of a major lipofuscin fluorophore in mice and humans with ABCR-mediated retinal and macular degeneration. Proc Natl Acad Sci USA. 2000; 97:7154-7159; Weng J, et al.; Cell. 1999; 98:13-23; Mata N L, et al.; Invest. Ophthalmol. Visual Sci. 2001; 42:1685-1690. All samples were analyzed by HPLC using absorbance and fluorescence detection. In these analyses, a column thermostat was employed to maintain the solvent and column temperature at 40° C. Identity of the indicated compounds was confirmed by on-line spectral analysis and by co-elution with authentic standards.

Correlation between Serum Retinol, Ocular Retinoids, and A2E

The data presented in Example 22 (FIG. 12) demonstrates a direct correlation between reduction in serum retinol and a reduction in the level of retinoids and the level of A2E in the eyecups of mammals. Notably serum retinol reduction tracks, in a dose-dependent manner, both ocular retinoid levels and ocular A2E levels. For example, fenretinide not only lowered serum retinol levels in mammals, but in addition, such a reduction of serum retinol effected the level of materials (e.g., A2E) associated with retinopathy and macular degenerations/dystrophies. Accordingly, agents, such as fenretinide, that cause serum retinol reductions also can be used to reduce A2E and retinoid levels in the eye, and further, be used to treat lipofuscin-based retinal diseases, e.g., retinopathies and macular degenerations/dystrophies, in the mammal.

### Example 23

### Validation of RBP as a Therapeutic Target for Arresting Accumulation of A2E

A non-pharmacological means of reducing lipofuscin fluorophores has been explored in order to validate our therapeutic approach based upon reduction of RBP levels in a patient. In this study, RBP protein levels have been reduced through genetic manipulation. Two new lines of mice expressing heterozygous mutations in retinol binding protein (RBP4) have been generated. The first line carries a heterozygous mutation only at the RBP locus (RBP+/−); the second line carries heterozygous mutations at both ABCA4 and RBP loci (ABCA4+/−/RBP4+/−). Thus, both lines demonstrate a ˜50% reduction in RBP expression and serum retinol. The RBP+/− mice will be wild type at the ABCA4 locus and, therefore, do not accumulate excessive amounts of A2E fluorophores. However, ABCA4+/− mice will accumulate A2E fluorophores at levels which are approximately 50% of that observed in ABCA4−/− (null homozygous) mice. At issue is whether the reduced expression of RBP in the ABCA4+/−/RBP+/− mice will have an effect on the accumulation of A2E fluorophores.

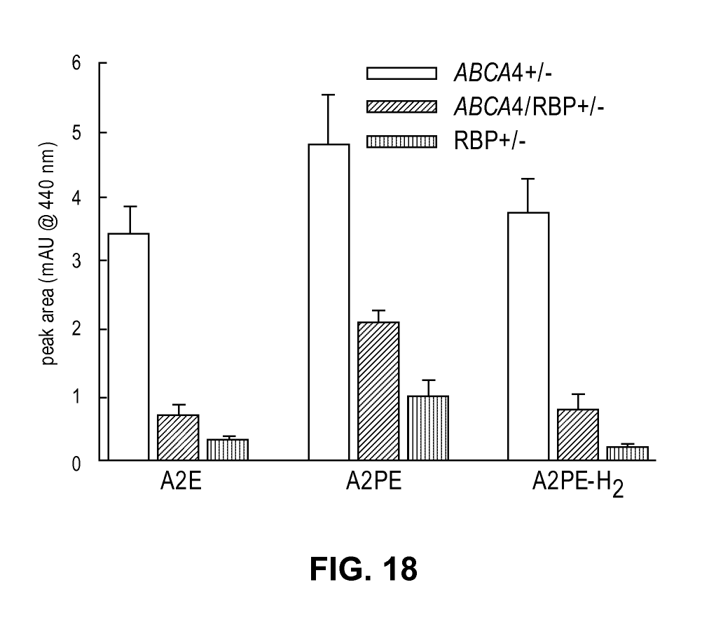

The levels of A2E and precursor fluorophores (A2PE and A2PE-H₂) in these mice have been monitored monthly over a three month period and compared to the fluorophore levels in ABCA4+/− mice. The data provide fluorophore levels in the three lines of mice at three months of age (FIG. 18). Overall, the ABCA4+/−/RBP+/− mice demonstrate a ˜70% reduction in total fluorophore level relative to the levels present in ABCA4+/− mice. In fact, the measured fluorophore levels in the ABCA4+/−/RBP+/− mice approach that observed in RBP+/− mice. These data validate RBP as a therapeutic target for reducing fluorophore levels in the eye. Further, these data demonstrate that agents or methods that inhibit the transcription or translation of RBP in a patient will also (a) reduce serum retinol levels in that patient, and (b) provide a therapeutic benefit in the retinol-related diseases described herein. Further, agents or methods that enhance the clearance of RBP in a patient will also produce such effects and benefits.

All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-21. (canceled)

22. A method for the treatment of Stargardt disease, comprising reducing serum retinol levels by at least 20% relative to pre-treatment levels in a human.

23. The method of claim 22, wherein the serum retinol levels are reduced by at least 50% relative to pre-treatment levels.

24. The method of claim 22, wherein the reduction of serum retinol levels is maintained for at least 6 months.

25. The method of claim 22, wherein the reduction of serum retinol levels is maintained for at least one year.

26. The method of claim 22, comprising administering to the subject a therapeutically-effective amount of a compound having the structure: 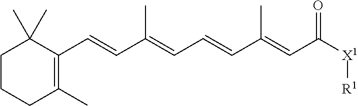
wherein
X¹ is selected from the group consisting of NR², O, S, CHR²;
R¹ is (CHR²)ₓ-L¹-R³, wherein
x is 0, 1, 2, or 3;
L¹ is a single bond or —C(O)—;
R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety selected from the group consisting of (C₂-C₇)alkenyl, (C₂-C₇)alkynyl, aryl, (C₃-C₇)cycloalkyl, (C₅-C₇)cycloalkenyl, and a heterocycle; wherein the moiety is optionally substituted with 1-3 substituents independently selected from the group consisting of halogen, OH, O(C₁-C₄)alkyl, NH(C₁-C₄)alkyl, O(C₁-C₄)fluoroalkyl, and N[(C₁-C₄)alkyl]₂; or a pharmaceutically acceptable salt thereof; provided that R³ is not H when both x is 0 and L¹ is a single bond.

27. The method of claim 26, wherein X¹ is NH and R³ is an aryl which has one substituent, wherein the substituent is a moiety selected from the group consisting of halogen, OH, O(C₁-C₄)alkyl, NH(C₁-C₄)alkyl, O(C₁-C₄)fluoroalkyl, and N[(C₁-C₄)alkyl]₂.

28. The method of claim 26, wherein the compound is N-(4-hydroxyphenyl)retinamide (HPR) or N-(4-methoxyphenyl)retinamide (MPR).

29. The method of claim 28, wherein the compound is N-(4-hydroxyphenyl)retinamide (HPR).

30. The method of claim 26, wherein the compound is systemically administered.

31. The method of claim 26, wherein the compound is administered orally.

32. The method of claim 26, further comprising administering at least one additional agent selected from the group consisting of an agent that reduces Retinol Binding Protein levels in the human, an agent that reduces Transerythrin levels in the human, an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, a resveratrol, a trans-stilbene compound, and 13-cis-retinoic acid.

33. A method for the treatment of a human carrying mutant ABCA4 gene, comprising reducing serum retinol levels by at least 20% relative to pre-treatment levels in a human.

34. The method of claim 33, wherein said human carrying mutant ABCA4 gene has diseases or conditions comprising recessive retinitis pigmentosa, cone-rod dystrophy, recessive cone-rod dystrophy or non-exudative age-related muscular degeneration.

35. The method of claim 33, wherein the serum retinol levels are reduced by at least 50% relative to pre-treatment levels.

36. The method of claim 33, wherein the reduction of serum retinol levels is maintained for at least 6 months.

37. The method of claim 33, comprising administering to the subject a therapeutically-effective amount of a compound having the structure: 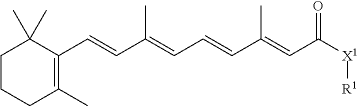
wherein
X¹ is selected from the group consisting of NR², O, S, CHR²;
R¹ is (CHR²)ₓ-L¹-R³, wherein
x is 0, 1, 2, or 3;
L¹ is a single bond or —C(O)—;
R² is a moiety selected from the group consisting of H, (C₁-C₄)alkyl, F, (C₁-C₄)fluoroalkyl, (C₁-C₄)alkoxy, —C(O)OH, —C(O)—NH₂, —(C₁-C₄)alkylamine, —C(O)—(C₁-C₄)alkyl, —C(O)—(C₁-C₄)fluoroalkyl, —C(O)—(C₁-C₄)alkylamine, and —C(O)—(C₁-C₄)alkoxy; and
R³ is H or a moiety selected from the group consisting of (C₂-C₇)alkenyl, (C₂-C₇)alkynyl, aryl, (C₃-C₇)cycloalkyl, (C₅-C₇)cycloalkenyl, and a heterocycle; wherein the moiety is optionally substituted with 1-3 substituents independently selected from the group consisting of halogen, OH, O(C₁-C₄)alkyl, NH(C₁-C₄)alkyl, O(C₁-C₄)fluoroalkyl, and N[(C₁-C₄)alkyl]₂; or a pharmaceutically acceptable salt thereof; provided that R³ is not H when both x is 0 and L¹ is a single bond.

38. The method of claim 37, wherein X¹ is NH and R³ is an aryl which has one substituent, wherein the substituent is a moiety selected from the group consisting of halogen, OH, O(C₁-C₄)alkyl, NH(C₁-C₄)alkyl, O(C₁-C₄)fluoroalkyl, and N[(C₁-C₄)alkyl]₂.

39. The method of claim 37, wherein the compound is N-(4-hydroxyphenyl)retinamide (HPR) or N-(4-methoxyphenyl)retinamide (MPR).

40. The method of claim 39, wherein the compound is N-(4-hydroxyphenyl)retinamide (HPR).

41. The method of claim 37, further comprising administering at least one additional agent selected from the group consisting of an agent that reduces Retinol Binding Protein levels in the human, an agent that reduces Transerythrin levels in the human, an inducer of nitric oxide production, an anti-inflammatory agent, a physiologically acceptable antioxidant, a physiologically acceptable mineral, a negatively charged phospholipid, a carotenoid, a statin, an anti-angiogenic drug, a matrix metalloproteinase inhibitor, a resveratrol, a trans-stilbene compound, and 13-cis-retinoic acid.
